(19) United States
(12) Patent Application Publication
(10) Pub. No.: US 2024/0218357 A1
(43) Pub. Date: Jul. 4, 2024

(54) ENGINEERED ONCOSELECTIVE PROTEIN EXPRESSION

(71) Applicant: Kernal Biologics, Inc., Cambridge, MA (US)

(72) Inventors: Yusuf Erkul, Cambridge, MA (US); Burak Yilmaz, Cambridge, MA (US); Cafer Ozdemir, Cambridge, MA (US)

(21) Appl. No.: 17/556,618

(22) PCT Filed: Jun. 19, 2020
(86) PCT No.: PCT/US2020/038742

§ 371 Date: Dec. 20, 2021

Related U.S. Application Data
(60) Provisional application No. 62/864,673, filed on Jun. 21, 2019.

(51) Int. Cl. C12N 15/11; A61K 31/7088

(52) U.S. Cl. CPC C12N 15/11; A61K 31/7088; C12N 2310/531

(57) ABSTRACT

The present disclosure provides technologies for achieving oncoselective translation. The use of two complementary pipelines to study the translation landscapes of cancer vs. normal cells described in this disclosure enables identification of oncoselective sequence motifs that can be used to engineer synthetic DNAs or mRNA constructs for cancer cell specific protein expression. The present disclosure describes the features of oncoselective motifs and provides embodiments of modular oncoselective construct designs that can be used to encode payloads of high therapeutic interest.

### CROSS REFERENCE TO RELATED APPLICATIONS

This application claims priority to U.S. Provisional Patent Application No. 62/864,673 filed on Jun. 21, 2019, the entire contents of which is hereby incorporated by reference.

### SEQUENCE LISTING

The instant application contains a Sequence Listing which has been submitted electronically in ASCII format and is hereby incorporated by reference in its entirety. Said ASCII copy, created on Jul. 24, 2020, is named 2013260-0006_SL.txt and is 16,831 bytes in size.

### BACKGROUND

There is a need to develop improved therapies for the treatment of cancer. Therapies tailored to specifically target cancer cells can provide an opportunity for unique treatment options.

### SUMMARY

The present invention provides technologies that achieve oncoselective expression of translatable nucleic acid sequences.

Delivery of nucleic acids for therapeutic purposes is a burgeoning and powerful field. Significant progress has recently been made in the field, including specifically with respect to technologies for stabilizing and/or effecting delivery of nucleic acids, particularly including translatable RNA molecules (e.g., mRNA).

Among other things, the present disclosure provides an insight that, notwithstanding these excellent developments and others, including the first marketing approval of an RNA therapeutic by the United States Food and Drug Administration, oncoselectivity remains a challenge. The present disclosure provides technologies for achieving oncoselective translation of nucleic acids, including those that are or deliver translatable RNAs (e.g., mRNAs). Among other things, the oncoselectivity achieved by the present disclosure permits use of certain therapeutic strategies (e.g., that may involve particularly toxic agents) that are unavailable and/or unadvisable (e.g., associated with an unacceptable risk profile) without such oncoselectivity, for example strategies that may have one or more undesirable effects in or on non-tumor cells and/or tissues.

The present disclosure provides technologies that achieve oncoselective expression of translatable nucleic acid sequences (e.g., in mRNAs). Among other things, the present disclosure defines sequence motifs that, when included in a translatable nucleic acid (e.g., an mRNA), achieve oncoselective expression of one or more encoded products. In particular, in some embodiments, the present disclosure provides oncoselective read-through motifs.

The present disclosure appreciates that studies have increasingly revealed alterations in ribosome structure and function that are associated with tumor development and/or progression. See, for example, Bastide and David Oncogenesis 2018 Apr. 7(4):34. The present disclosure further appreciates that such ribosomal alterations can be harnessed to improve cancer therapy. Among other things, the present disclosure teaches that oncoselective translational read-through can be utilized to achieve oncoselective expression and/or activity of a translation product (e.g., a polypeptide).

The present disclosure provides technologies for defining oncoselective translation sequence elements such as, for example, oncoselective read-through motifs, and furthermore provides defined such oncoselective translation sequences elements. Furthermore, the present disclosure provides a variety of insights relating to existing technologies that aim to achieve oncoselective expression and/or activity (e.g., via oncoselective delivery and/or expression) of a payload intended to be targeted to cancer cells.

For example, the present disclosure appreciates that many technologies described as “oncoselective” or “cancer cell specific” often achieve only modest discrimination between cancer and non-cancer contexts. For example, Wroblewska et al. used an mRNA circuit that is activated when intracellular miR-21 levels are high and miR-141, miR-142(3p) and miR-146a levels are low and obtained approximately only 6-fold higher in vitro cell killing in HeLa cells compared to HEK293 cells (Nat Biotechnol. 2015 Aug;33(8):839-41). Likewise, Jain et al., 2018 (Nucleic Acid Ther. 2018 Oct 1; 28(5): 285 96.) used miR-122 and miR-142 target site insertion to reduce in vivo mRNA activity by 89% and 85% (corresponding to a ˜6-10 fold decrease) in the liver and spleen, respectively. However, when intratumorally injected a significant portion of miRNA target siteinserted mRNAs were also shown to be taken up by healthy cells, including tumor infiltrating immune cells (Hewitt et al., Sci Transl Med. 2019 Jan 30;11(477)). This activity in immune cells can be counter-productive for immuno-oncology applications of mRNAs encoding cell killing proteins.

The present disclosure particularly identifies the source of a problem with certain existing technologies for assessing sequence elements that may contribute to translational read-through. Among other things, ribosome profiling has been used to infer translational read-through. However, ribosome profiling has biases for RNA sequences and structures that slow down or stall ribosomes. Therefore, those events can be overrepresented and result in inaccurate determination of the efficiency of read-through, when used alone. On the other hand, an LC/MS based proteomics approach, while free from RNA level artifacts, has a high false negative rate, because it can miss peptides with low flyability (i.e. low efficiency of ionization, transfer, and detection) or abundance. Thus, the present disclosure, among other things, describes novel uses of technologies to assess sequence elements that may contribute to translational read-through.

In some embodiments, the present disclosure considers translation to be “oncoselective” when translation preferably occurs in cancer cell(s) as compared with appropriate comparable non-cancer cell(s). For example, in some embodiments, translation may be considered to be oncoselective when it is observed to be at least two (2)-fold higher in cancer cell(s) as compared with appropriate comparable non-cancer cells; in some embodiments, oncoselective translation may be at least 3, 4, 5, 6, 7, 8, 9, 10, 15, 20 or more -fold higher in cancer cell(s) as compared with appropriately comparable non-cancer cells.

In some embodiments, oncoselective translation may be considered to be oncospecific (e.g., when translation is not detectable in appropriate comparable non-cancer cell(s), but is detectable in the relevant cancer cell(s).

In some embodiments, the present disclosure provides engineered nucleic acids whose nucleotide sequence include a sequence element that is (or is a complement of) an oncoselective translation sequence element. Thus, in some embodiments, the present disclosure provides technologies that are or deliver a translatable nucleic acid (e.g., an RNA, and specifically an mRNA) that includes an oncoselective translation sequence element as described herein and/or otherwise shows oncoselective translation of a payload sequence.

In some embodiments, the present disclosure provides an engineered nucleic acid whose nucleotide sequence includes a sequence element that is or is a complement of an oncoselective translation sequence element. In some embodiments, the engineered nucleic acid's nucleotide sequence includes an open reading frame or complement thereof. In some embodiments, the oncoselective translation sequence element is or comprises an oncoselective read-through motif within or upstream of the open reading frame. In some embodiments, the oncoselective readthrough motif comprises an upstream flanking sequence, a stop codon, and a downstream flanking sequence.

In some embodiments, an oncoselective readthrough motif comprises a sequence selected from the group comprising: VNNNNNNMNNMWK (SEQ ID NO: 24), NNNVWNNKGHHNH (SEQ ID NO: 25), DVHVNNNCWNNNB (SEQ ID NO: 26), MWBNNNNNNNNNN (SEQ ID NO: 27), WGNNSNHNHDNNN (SEQ ID NO: 28), VNNNNNNMNNMWK (SEQ ID NO: 29) or VMNNWNKNNNNNN (SEQ ID NO: 30), wherein V stands for A, C or G, M stands for A or C, W stands for A or T/U, K stands for G or T/U, H stands for A, C or T/U, D stands for A,G or T/U, B stands for C, G or T/U, S stands for G or C, N stands for any nucleotide, within the region that spans the readthrough stop codon and the first 14 nucleotides of the downstream flanking sequence. In some embodiments, the oncoselective read through motif comprises a stem loop; a bulge loop, a pseudoknot, or a combination thereof within the first 50 nucleotides of the downstream flanking sequence and part of this stem loop located preferably within stop codon and the first 16 nucleotides of the downstream flanking sequence, or a combination thereof. In some embodiments, the stem loop comprises more than 20 base paired nucleotides within first 50 nucleotides of the downstream flanking sequence.

In some embodiments, the oncoselective read through motif comprises a downstream flanking sequence with a GC content of more than 42%, more than 48%, preferably more than 54%. In some embodiments, an oncoselective read through motif comprises a codon that encodes proline residue. In some embodiments, the open reading frame of an engineered nucleic acid encodes a suicide protein. In some embodiments, the engineered nucleic acid has reduced immunogenicity.

In some embodiments, the present disclosure provides a nucleic acid whose sequence includes an open reading frame, or a complement thereof, into or before which an oncoselective read-through motif has been engineered, wherein the open reading frame encodes a payload protein selected from the group consisting of a suicide protein, cell surface antigen, an antibody agent, a toxin, a genetic modification protein, or a viral replication protein. In some embodiments, the present disclosure provides, a pharmaceutical composition comprising an engineered nucleic acid whose nucleotide sequence includes a sequence element that is or is a complement of an oncoselective translation sequence element. In some embodiments, the pharmaceutical composition comprises nanoparticles. In some embodiments, the engineered nucleic acid is expressed in a cell so that administration of the pharmaceutical composition delivers RNA to the cell.

In some embodiments, the present disclosure provides a method of treating cancer in a subject, wherein the method comprises administering a therapeutically effective amount of an engineered nucleic acid whose nucleotide sequence includes a sequence element that is or is a complement of an oncoselective translation sequence element or a pharmaceutical composition comprising the engineered nucleic acid. In some embodiments, the cancer in the treated subject comprises oncogenic ribosomes. In some embodiments, the oncogenic ribosomes comprise at least one of loss of p53 activity, loss of RB activity, FBL overexpression, or hemizygous loss of ribosomal protein genes.

In some embodiments, the present disclosure provides an oncoselective translation sequence element comprising a read-through consensus sequence, sequence with high G-C content; a codon encoding proline; a stem loop; a bulge loop, a pseudoknot or a combination thereof. In some embodiments, the present disclosure provides a method of identifying an onco-selective nucleic acid sequences the method comprising transcriptome-wide translatome analysis. In some embodiments, the present disclosure provides a method of engineering oncoselective nucleic acids by inserting a readthrough motif within or before the open reading frame.

### BRIEF DESCRIPTION OF THE DRAWING

FIG. 1 is a schematic representation of an exemplary onco-selective readthrough motif. Each motif comprises an upstream flanking sequence, a stop codon, and a downstream flanking sequence. The upstream flanking sequence (approximately 60 nucleotides) has high GC content within 3^{rd} (wobble) positions of the codons. The downstream flanking sequence (approximately 50 nucleotides) has high GC content, a linear consensus sequence within the first 10-12 nucleotides, and a stem loop structure (or another stable RNA structure).

FIG. 2 is a construct map of U1n through U10n. Each construct contains a 7-methylGuanosine cap, 5′UTR, a coding region, a 3′UTR and a poly-A tail (post-transcriptionally added). The coding region comprises a start codon (ATG/AUG), a readthrough (RT) motif, a self-cleaving peptide and NanoLuciferase coding region (open reading frame) lacking the first ATG/AUG). This modular design allows for swift replacement of the encoded gene, while preserving the vector backbone and the RT motif.

FIG. 3 shows translational activity of tested constructs. Healthy (HUVEC) and cancer (NCI-H1299) cells were seeded 24 hours prior to transfection. Lipofectamine formulated constructs (U1n-U10n) were incubated for 15 minutes at room temperature and then were added to the cell culture medium. Nanoluciferase activity was measured 16 hours later. Transfections were performed in triplicates and data is shown as mean +/−SD.

FIG. 4 shows secondary structure of an exemplary readthrough motif. Readthrough motif surrounding the stop codon of U2n construct includes a large stem loop structure with internal loops and bulges. Figure discloses SEQ ID NOS 2 and 31, respectively, in order of appearance.

FIG. 5 is a construct map of Onco-333. The design of Onco-333 includes a cap structure, a 5′UTR sequence, a firefly Luciferase (fLuc) coding region containing a readthrough motif, a 3′UTR sequence and a poly-A tail (posttranscriptionally added).

FIG. 6 shows in vitro testing of Onco-333 mRNA. Onco-333 mRNA was transfected into healthy cells (BJ fibroblasts), transformed cells (HEK293 cells expressing Ad E1A and E1B, which suppress Rb and p53 function), and leukemia cells (K562), demonstrating no detectable fLuc activity in healthy cells. Luciferase activity was measured by Bright Glo Assay (Promega). Transfections were performed in triplicates and data is depicted as mean +/−SD.

FIG. 7 shows in vivo testing of Onco-333 mRNA. Onco-333 mRNA and wild type mRNA (control mRNA) were formulated with TransIT mRNA reagent and injected (I.V.) into healthy C57/B16 mice. 24 hours later fLuc activity levels were measured in liver, spleen, bone marrow (BM), and lung tissue homogenates ex vivo, demonstrating that Onco-333 is also onco-selective in vivo (n=3 per group).

FIG. 8 shows Onco-333 activity in human and mouse leukemia and in lung cancer cell lines with TP53 mutation. Murine non-small cell lung cancer cells (LL/2), human non-small cell lung cancer cells (NCI-H1299), human acute myeloid leukemia (HL-60), murine acute myeloid leukemia (C1498) cells were transfected with Onco-333 mRNA and fLuc activity was measured 24 hours later. (Experiments were performed in triplicates and data is shown as mean +/−SD). Mutation statuses of TP53 are as follows: LL2: c.G1001C:p.R334P, H1299: homozygous c.(del) , HL60: homozygous c.(del).

FIG. 9 demonstrates analysis of sequence features found within 3′UTR sequences of the readthrough transcripts. Healthy and Cancer RT transcripts were analyzed using position weight matrices (PWM), which assumes that the sequence features are located at the same exact nucleotide positions. Analytical foreground contained RT transcripts' and background contained the rest of the human transcriptome's first 120 nucleotides from 3′UTR. Stop codons (amber, ochre and opal) were perfectly aligned. In addition, analysis of first 120 nucleotides of 3′UTR sequences of the RT mRNA transcripts showed GC rich sequences. Red horizontal lines are the p<0.05 significance line for over or under representations. As shown on the left, healthy RT transcripts do not have a significant trend. On the other hand, cancer transcripts have G-C overrepresentation and AU underrepresentation within the first 48-50 nucleotides.

FIG. 10 demonstrates analysis of sequence features found within coding region sequences of the readthrough transcripts. In addition to the initial region of 3′UTR, the last nucleotides of coding sequence (CDS) region of cancer RT transcripts have also some differential tendency that are not observed in healthy RT transcripts. As shown, cancer specific RT transcripts with TAG and TGA stop codons have overrepresentation of G/C nucleotides at the wobble bases. Stronger secondary structures require higher G/C nucleotide content, but the coding region has codon constraints for encoding the correct protein with the first two position in most codons being fixed.

FIG. 11 demonstrates base pairing coverage analysis. In order to analyze the secondary structure of the RNA motif, cancer RT and healthy RT transcripts were folded as whole mRNAs via Co-fold algorithm. Dot bracket notation of the region around the stop codon (100 nucleotides in CDS including stop codon, 100 nt in 3′UTR) for cancer and healthy RT transcripts were extracted. Coverage analysis was performed by identifying the number of base pairings (y-axis) arching over each nucleotide position (x-axis). Orange line shows healthy, blue line shows cancer RT transcripts. This analysis shows that stop codon readthrough requires more structured regions around stop codon. The stop codon is located at positions 2-4 on the X axis. Cancer transcripts are more structured around the stop codon and the first 16 nucleotides of the 3′UTR.

FIGS. 12 and 13 show linear consensus sequences within the onco-selective readhthrough transcripts. First 13 nucleotides of the 3′UTR regions of Healthy and Cancer RT transcripts with ochre were analyzed via GLAM2 (Gapped Local Alignment of Motifs), which does not assume that the sequence features are located at the same exact nucleotide positions. This analysis revealed the following consensus sequences: VNNNNNNMNNMWK (SEQ ID NO: 24), NNNVWNNKGHHNH (SEQ ID NO: 25), DVHVNNNCWNNNB (SEQ ID NO: 26), MWBNNNNNNNNNN (SEQ ID NO: 27), WGNNSNHNHDNNN (SEQ ID NO: 28), VNNNNNNMNNMWK (SEQ ID NO: 29) or VMNNWNKNNNNNN (SEQ ID NO: 30), where in V stands for A, C or G, M stands for A or C, W stands for A or T/U, K stands for G or T/U, H stands for A, C or T/U, D stands for A,G or T/U, B stands for C, G or T/U, S stands for G or C, N stands for any nucleotide. FIG. 12 discloses SEQ ID NOS 32 and 24, respectively, in order of appearance.

FIGS. 14 and 15 demonstrate comparative analysis of onco-selective readthrough transcripts with different stop codons using deep learning. A feedforward deep neural network (fully connected autoencoder) model was built and trained with first 13 nucleotides of the 3′UTR regions of cancer readthrough transcripts to analyze similarities of the readthrough sequence succeeding UAG (Amber), UAA (Ochre), and UGA (Opal) codons. We then generated 2-D and 3-D images from principle component analyses, which showed that Amber and ochre transcripts have very tight clusters in the latent space while UGA has spread representation. This correlates with the efficiency of stop codons which has been reported in the literature.

FIG. 16 further demonstrates the Onco-333 activity in cells with or without p53 inhibition via heat sensitive SV40 large T-antigen. Cells grown at 32° C. have a functional SV40, which inhibits p53 activity, have high Onco-333 expression compared to cells grown at 39° C., which de-inhibits p53.

FIGS. 17A and 17B further demonstrates oncoselective expression of two reporter payloads using oncoselective motifs described in the present disclosure.

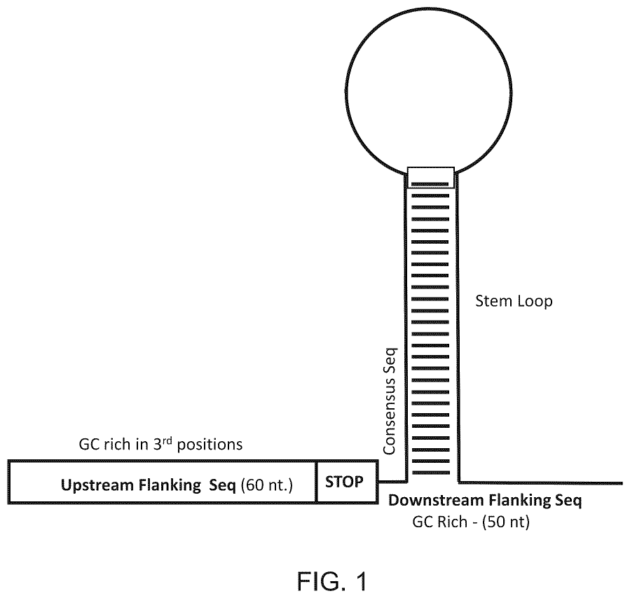

### DEFINITIONS

Administration: As used herein, the term “administration” typically refers to the administration of a composition to a subject or system. Those of ordinary skill in the art will be aware of a variety of routes that may, in appropriate circumstances, be utilized for administration to a subject, for example a human. For example, in some embodiments, administration may be systemic or local. In some embodiments, administration may be enteral or parenteral. In some embodiments, administration may be by injection (e.g., intramuscular, intravenous, or subcutaneous injection). In some embodiments, injection may involve bolus injection, drip, perfusion, or infusion. In some embodiments administration may be topical. Those skilled in the art will be aware of appropriate administration routes for use with particular therapies described herein, for example from among those listed on www.fda.gov, which include auricular (otic), buccal, conjunctival, cutaneous, dental, endocervical, endosinusial, endotracheal, enteral, epidural, extra-amniotic, extracorporeal, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal, intracorporus cavernosum, intradermal, intradiscal, intraductal, intraduodenal, intradural, intraepidermal, intraesophageal, intragastic, intragingival, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumoral, intratympanic, intrauterine, intravascular, intravenous, intravenous bolus, intravenous drip, intraventricular, intravitreal, laryngeal, nasal, nasogastric, ophthalmic, oral, oropharyngeal, parenteral, percutaneous, periarticular, peridural, perineural, periodontal, rectal, respiratory (e.g., inhalation), retrobulbar, soft tissue, subarachnoid, subconjunctival, subcutaneous, sublingual, submucosal, topical, transdermal, transmucosal, transplacental, transtracheal, ureteral, urethral, or vaginal. In some embodiments, administration may involve electro-osmosis, hemodialysis, infiltration, iontophoresis, irrigation, and/or occlusive dressing. In some embodiments, administration may involve dosing that is intermittent (e.g., a plurality of doses separated in time) and/or periodic (e.g., individual doses separated by a common period of time) dosing. In some embodiments, administration may involve continuous dosing.

Agent: As used herein, the term “agent”, may refer to a compound, molecule, or entity of any chemical class including, for example, a small molecule, polypeptide, nucleic acid, saccharide, lipid, metal, or a combination or complex thereof. In some embodiments, the term “agent” may refer to a compound, molecule, or entity that comprises a polymer. In some embodiments, the term may refer to a compound or entity that comprises one or more polymeric moieties. In some embodiments, the term “agent” may refer to a compound, molecule, or entity that is substantially free of a particular polymer or polymeric moiety. In some embodiments, the term may refer to a compound, molecule, or entity that lacks or is substantially free of any polymer or polymeric moiety.

Amino acid: As used herein, the term “amino acid” refers to any entity that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. As used herein, the term “standard amino acid” refers to any of the twenty L-amino acids commonly found in naturally occurring peptides. “Nonstandard amino acid” refers to any amino acid, other than the standard amino acids, regardless of whether it is or can be found in a natural source. In some embodiments, an amino acid, including a carboxy- and/or amino-terminal amino acid in a polypeptide, can contain a structural modification as compared to the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared to the general structure. In some embodiments, such modification may, for example, alter the stability or the circulating half-life of a polypeptide containing the modified amino acid as compared to one containing an otherwise identical unmodified amino acid. In some embodiments, such modification does not significantly alter a relevant activity of a polypeptide containing the modified amino acid, as compared to one containing an otherwise identical unmodified amino acid. As will be clear from context, in some embodiments, the term “amino acid” may be used to refer to a free amino acid; in some embodiments it may be used to refer to an amino acid residue of a polypeptide, e.g., an amino acid residue within a polypeptide.

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present invention include glycosylated Fc domains, including Fc domains with modified or engineered such glycosylation. For purposes of the present invention, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc, as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, embodiments, an antibody utilized in accordance with the present invention is in a format selected from, but not limited to, intact IgA, IgG, IgE or IgM antibodies; bi- or multi- specific antibodies (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; NanobodiesR minibodies; BiTE®s; ankyrin repeat proteins or DARPINS®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.]

Antibody agent: As used herein, the term “antibody agent” refers to an agent that specifically binds to a particular antigen. In some embodiments, the term encompasses any polypeptide or polypeptide complex that includes immunoglobulin structural elements sufficient to confer specific binding. Exemplary antibody agents include, but are not limited to monoclonal antibodies or polyclonal antibodies. In some embodiments, an antibody agent may include one or more constant region sequences that are characteristic of mouse, rabbit, primate, or human antibodies. In some embodiments, an antibody agent may include one or more sequence elements are humanized, primatized, chimeric, etc, as is known in the art. In many embodiments, the term “antibody agent” is used to refer to one or more of the art-known or developed constructs or formats for utilizing antibody structural and functional features in alternative presentation. For example, embodiments, an antibody agent utilized in accordance with the present invention is in a format selected from, but not limited to, intact IgA, IgG, IgE or IgM antibodies; bi- or multi- specific antibodies (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies: masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINS®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.]. In many embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments an included CDR is substantially identical to a reference CDR in that it shows at least 96%, 96%, 97%, 98%, 99%, or 100% sequence identity with the reference CDR. In some embodiments an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments an included CDR is substantially identical to a reference CDR in that at least one amino acid within the included CDR is substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain.

Cancer: As used herein, the term “cancer” refers to a disease, disorder, or condition in which cells exhibit relatively abnormal, uncontrolled, and/or autonomous growth, so that they display an abnormally elevated proliferation rate and/or aberrant growth phenotype characterized by a significant loss of control of cell proliferation. In some embodiments, a cancer may be characterized by one or more tumors. Those skilled in the art are aware of a variety of types of cancer including, for example, adrenocortical carcinoma, astrocytoma, basal cell carcinoma, carcinoid, cardiac, cholangiocarcinoma, chordoma, chronic myeloproliferative neoplasms, craniopharyngioma, ductal carcinoma in situ, ependymoma, intraocular melanoma, gastrointestinal carcinoid tumor, gastrointestinal stromal tumor (GIST), gestational trophoblastic disease, glioma, histiocytosis, leukemia (e.g., acute lymphoblastic leukemia (ALL), acute myeloid leukemia (AML), chronic lymphocytic leukemia (CLL), chronic myelogenous leukemia (CML), hairy cell leukemia, myelogenous leukemia, myeloid leukemia), lymphoma (e.g., Burkitt lymphoma [non-Hodgkin lymphoma], cutaneous T-cell lymphoma, Hodgkin lymphoma, mycosis fungoides, Sezary syndrome, AIDS-related lymphoma, follicular lymphoma, diffuse large B-cell lymphoma), melanoma, merkel cell carcinoma, mesothelioma, myeloma (e.g., multiple myeloma), myelodysplastic syndrome, papillomatosis, paraganglioma, pheochromacytoma, pleuropulmonary blastoma, retinoblastoma, sarcoma (e.g., Ewing sarcoma, Kaposi sarcoma, osteosarcoma, rhabdomyosarcoma, uterine sarcoma, vascular sarcoma), Wilms' tumor, and/or cancer of the adrenal cortex, anus, appendix, bile duct, bladder, bone, brain, breast, bronchus, central nervous system, cervix, colon, endometrium, esophagus, eye, fallopian tube, gall bladder, gastrointestinal tract, germ cell, head and neck, heart, intestine, kidney (e.g., Wilms' tumor), larynx, liver, lung (e.g., non-small cell lung cancer, small cell lung cancer), mouth, nasal cavity, oral cavity, ovary, pancreas, rectum, skin, stomach, testes, throat, thyroid, penis, pharynx, peritoneum, pituitary, prostate, rectum, salivary gland, ureter, urethra, uterus, vagina, or vulva. In some embodiments, a cancer may be or comprise one or more solid tumors. In some embodiments, a cancer may be or comprise one or more haematologic tumors.

Combination therapy: As used herein, the term “combination therapy” refers to a clinical intervention in which a subject is simultaneously exposed to two or more therapeutic regimens (e.g. two or more therapeutic agents). In some embodiments, the two or more therapeutic regimens may be administered simultaneously. In some embodiments, the two or more therapeutic regimens may be administered sequentially (e.g., a first regimen administered prior to administration of any doses of a second regimen). In some embodiments, the two or more therapeutic regimens are administered in overlapping dosing regimens. In some embodiments, administration of combination therapy may involve administration of one or more therapeutic agents or modalities to a subject receiving the other agent(s) or modality. In some embodiments, combination therapy does not necessarily require that individual agents be administered together in a single composition (or even necessarily at the same time). In some embodiments, two or more therapeutic agents or modalities of a combination therapy are administered to a subject separately, e.g., in separate compositions, via separate administration routes (e.g., one agent orally and another agent intravenously), and/or at different time points. In some embodiments, two or more therapeutic agents may be administered together in a combination composition, or even in a combination compound (e.g., as part of a single chemical complex or covalent entity), via the same administration route, and/or at the same time.

Comparable: As used herein, the term “comparable” refers to two or more agents, entities, situations, sets of conditions, etc. that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Corresponding to: As used herein in the context of polypeptides, nucleic acids, and chemical compounds, the term “corresponding to”, designates the position/identity of a structural element, e.g., of an amino acid residue, a nucleotide residue, or a chemical moiety, in a compound or composition through comparison with an appropriate reference compound or composition. For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, those of ordinary skill will appreciate that, for purposes of simplicity, residues in a polypeptide are often designated using a canonical numbering system based on a reference related polypeptide, so that an amino acid “corresponding to” a residue at position 190, for example, need not actually be the 190^{th} amino acid in a particular amino acid chain but rather corresponds to the residue found at position 190 in the reference polypeptide; those of ordinary skill in the art readily appreciate how to identify “corresponding” amino acids (see. e.g., Benson et al. Nucl. Acids Res. (1 Jan. 2013) 41 (D1): D36-D42; Pearson et al. PNAS Vol.85, pp. 2444-2448, April 1988). Those skilled in the art will be aware of various sequence alignment strategies, including software programs such as, for example, BLAST, CS-BLAST, CUSASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE that can be utilized, for example, to identify “corresponding” residues in polypeptides and/or nucleic acids in accordance with the present disclosure.

Expression: As used herein, the term “expression” of a nucleic acid sequence refers to the generation of a gene product from the nucleic acid sequence. In some embodiments, a gene product can be a transcript (e.g., a primary transcript or a processed transcript such as an mRNA). In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Flanking Sequence: As used herein the term “flanking sequence” refers to any sequence that precedes or succeeds a sequence or domain of interest. For example, a region upstream of a stop codon can be referred to as “upstream flanking region”.

Gene: As used herein, the term “gene” refers to a DNA or RNA sequence that encodes a gene product (e.g., an RNA product and/or a polypeptide product). In some embodiments, a gene includes a coding sequence (e.g., a sequence that encodes a particular gene product); in some embodiments, a gene includes a non-coding sequence. In some particular embodiments, a gene may include both coding (e.g., exonic) and non-coding (e.g., intronic) sequences. In some embodiments, a gene may include one or more regulatory elements (e.g. promoters, enhancers, silencers, termination signals) that, for example, may control or impact one or more aspects of gene expression (e.g., cell-type-specific expression, inducible expression). In some embodiments, a gene is located or found (or has a nucleotide sequence identical to that located or found) in a genome (e.g., in or on a chromosome or other replicable nucleic acid).

Mutant: As used herein, the term “mutant” refers to an organism, a cell, or a biomolecule (e.g., a nucleic acid or a polypeptide) that has a genetic variation as compared to a reference organism, cell, or biomolecule. For example, a mutant nucleic acid or polypeptide may, in some embodiments, have, for example, a substitution of one or more residues (e.g., of one or more nucleobases or amino acids), a deletion of one or more residues (e.g., an internal deletion or a truncation), an insertion of one or more residues, an inversion of two or more residues, etc, as compared to a reference nucleic acid molecule. Those skilled in the art will be familiar with various particular types of such nucleic acid or polypeptide mutants—e.g., fusions, indels, etc. An organism or cell comprising or expressing a mutant nucleic acid or polypeptide is also sometimes referred to herein as a “mutant.” In some embodiments, a mutant comprises a genetic variant that is associated with a loss of function of a gene product. A loss of function may be a complete abolishment of function, e.g., an abolishment of activity (e.g., of bindig activity, enzymatic activity, etc), or a partial loss of function, e.g., a diminished activity (e.g., binding activity, enzymatic activity, etc). In some embodiments, a mutant comprises a genetic variant that is associated with a gain of function, e.g., with enhancement of an existing activity, or gain of a new activity relative to an appropriate reference (e.g., the same entity absent the genetic variation). In some embodiments, a gain of function mutant may have gained an alteration in a characteristic or activity. In some embodiments, a gain of function mutant may have constitutive activity. In some embodiments, a loss of function mutant may have lost (or reduced relative to a reference) a desirable activity. In some embodiments, the reference organism, cell, or biomolecule relative to which a mutant's structure, level, and/or activity is compared, is a wild-type organism, cell, or biomolecule.

Nucleic acid: As used herein, the term “nucleic acid” refers to a polymer of at least three nucleotides. In some embodiments, a nucleic acid is or comprises DNA. In some embodiments, a nucleic acid is or comprises RNA. In some embodiments, a nucleic acid is single stranded. In some embodiments, a nucleic acid is double stranded. In some embodiments, a nucleic acid comprises both single and double stranded portions. In some embodiments, a nucleic acid comprises a backbone that comprises one or more phosphodiester linkages. In some embodiments, a nucleic acid comprises a backbone that comprises both phosphodiester and non-phosphodiester linkages. For example, in some embodiments, a nucleic acid may comprise a backbone that comprises one or more phosphorothioate or 5′-N-phosphoramidite linkages and/or one or more peptide bonds, e.g., as in a “peptide nucleic acid”. In some embodiments, a nucleic acid comprises one or more, or all, natural residues (e.g., adenine, cytosine, deoxyadenosine, deoxycytidine, deoxyguanosine, deoxythymidine, guanine, thymine, uracil). In some embodiments, a nucleic acid comprises on or more, or all, non-natural residues. In some embodiments, a non-natural residue comprises a nucleoside analog (e.g., 2-aminoadenosine, 2-thiothymidine, inosine, pyrrolo-pyrimidine, 3 -methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5 -propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a non-natural residue comprises one or more modified sugars (e.g., 2′-fluororibose, ribose, 2′-deoxyribose, arabinose, and hexose) as compared to those in natural residues. In some embodiments, a nucleic acid has a nucleotide sequence that encodes a functional gene product such as an RNA or polypeptide. In some embodiments, a nucleic acid has a nucleotide sequence that comprises one or more introns. In some embodiments, a nucleic acid may be prepared by isolation from a natural source, enzymatic synthesis (e.g., by polymerization based on a complementary template, e.g., in vivo or in vitro, reproduction in a recombinant cell or system, or chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 1 10, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long.

Peptide: As used herein, the term “peptide” refers to a polypeptide that is typically relatively short, for example having a length of less than about 100 amino acids, less than about 50 amino acids, less than about 40 amino acids less than about 30 amino acids, less than about 25 amino acids, less than about 20 amino acids, less than about 15 amino acids, or less than 10 amino acids.

Pharmaceutical composition: As used herein, the term “pharmaceutical composition” refers to a composition that is suitable for administration to a human or animal subject. In some embodiments, a pharmaceutical composition comprises an active agent formulated together with one or more pharmaceutically acceptable carriers. In some embodiments, the active agent is present in a unit dose amount appropriate for administration in a therapeutic regimen. In some embodiments, a therapeutic regimen comprises one or more doses administered according to a schedule that has been determined to show a statistically significant probability of achieving a desired therapeutic effect when administered to a subject or population in need thereof. In some embodiments, a pharmaceutical composition may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces. In some embodiments, a pharmaceutical composition is intended and suitable for administration to a human subject. In some embodiments, a pharmaceutical composition is sterile and/or substantially pyrogen-free.

Polypeptide: As used herein, the term “polypeptide”, refers to a polymer of at least three amino acid residues. In some embodiments, a polypeptide comprises one or more, or all, natural amino acids. In some embodiments, a polypeptide comprises one or more, or all non-natural amino acids. In some embodiments, a polypeptide comprises one or more, or all, D-amino acids. In some embodiments, a polypeptide comprises one or more, or all, L-amino acids. In some embodiments, a polypeptide comprises one or more pendant groups or other modifications, e.g., modifying or attached to one or more amino acid side chains, at the polypeptide's N-terminus, at the polypeptide's C-terminus, or any combination thereof. In some embodiments, a polypeptide comprises one or more modifications such as acetylation, amidation, aminoethylation, biotinylation, carbamylation, carbonylation, citrullination, deamidation, deimination, eliminylation, glycosylation, lipidation, methylation, pegylation, phosphorylation, sumoylation, or combinations thereof. In some embodiments, a polypeptide may participate in one or more intra- or inter-molecular disulfide bonds. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may comprise a stapled polypeptide. In some embodiments, a polypeptide participates in non-covalent complex formation by non-covalent or covalent association with one or more other polypeptides (e.g., as in an antibody). In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a useful polypeptide may comprise a fragment of a parent polypeptide. In some embodiments, a useful polypeptide as may comprise a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Reference: As used herein, the term “reference” refers to a standard or control relative to which a comparison is performed. For example, in some embodiments, an agent, animal, individual, population, sample, sequence, or value of interest is compared to a reference or control agent, animal, individual, population, sample, sequence, or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Sample: As used herein, the term “sample” refers to a biological sample obtained or derived from a source of interest, as described herein. In some embodiments, a source of interest is or comprises an organism, such as a microbe, a plant, an animal or a human. In some embodiments, a biological sample is or comprises biological tissue or fluid, or one or more components thereof. In some embodiments, a biological sample may be or comprise bone marrow; blood; blood cells; ascites; tissue or biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; other body fluids, secretions, and/or excretions; and/or cells therefrom. In some embodiments, a biological sample comprises cells obtained from an individual, e.g., from a human or animal subject. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces). In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or polypeptides extracted from a sample or obtained by subjecting a primary sample to techniques such as amplification or reverse transcription of mRNA, isolation and/or purification of certain components.

Subject: As used herein, the term “subject” refers to an organism, for example, a mammal (e.g., a human, a non-human mammal, a non-human primate, a primate, a laboratory animal, a mouse, a rat, a hamster, a gerbil, a cat, a dog). In some embodiments a human subject is an adult, adolescent, or pediatric subject. In some embodiments, a subject is suffering from a disease, disorder or condition, e.g., a disease, disorder or condition that can be treated as provided herein, e.g., a cancer or a tumor listed herein. In some embodiments, a subject is susceptible to a disease, disorder, or condition; in some embodiments, a susceptible subject is predisposed to and/or shows an increased risk (as compared to the average risk observed in a reference subject or population) of developing the disease, disorder or condition. In some embodiments, a subject displays one or more symptoms of a disease, disorder or condition. In some embodiments, a subject does not display a particular symptom (e.g,. clinical manifestation of disease) or characteristic of a disease, disorder, or condition. In some embodiments, a subject does not display any symptom or characteristic of a disease, disorder, or condition. In some embodiments, a subject is a patient. In some embodiments, a subject is an individual to whom diagnosis and/or therapy is and/or has been administered.

Therapeutic agent: As used herein, the term “therapeutic agent” generally refers to an agent that elicits a desired effect (e.g., a desired biological, clinical, or pharmacological effect) when administered to a subject. In some embodiments, an agent is considered to be a therapeutic agent if it demonstrates a statistically significant effect across an appropriate population. In some embodiments, an appropriate population is a population of subjects suffering from and/or susceptible to a disease, disorder or condition. In some embodiments, an appropriate population is a population of model organisms. In some embodiments, an appropriate population may be defined by one or more criterion such as age group, gender, genetic background, preexisting clinical conditions, prior exposure to therapy. In some embodiments, a therapeutic agent is a substance that alleviates, ameliorates, relieves, inhibits, prevents, delays onset of, reduces severity of, and/or reduces incidence of one or more symptoms or features of a disease, disorder, and/or condition in a subject when administered to the subject in an effective amount. In some embodiments, a “therapeutic agent” is an agent that has been or is required to be approved by a government agency before it can be marketed for administration to humans. In some embodiments, a “therapeutic agent” is an agent for which a medical prescription is required for administration to humans. In some embodiments, therapeutic agents may be CREBBP antagonists as described herein.

Therapeutically effective amount: As used herein, the term “therapeutically effective amount” refers to an amount that produces a desired effect (e.g., a desired biological, clinical, or pharmacological effect) in a subject or population to which it is administered. In some embodiments, the term refers to an amount statistically likely to achieve the desired effect when administered to a subject in accordance with a particular dosing regimen (e.g., a therapeutic dosing regimen). In some embodiments, the term refers to an amount sufficient to produce the effect in at least a significant percentage (e.g., at least about 25%, about 30%, about 40%, about 50%, about 60%, about 70%, about 80%, about 90%, about 95%, or more) of a population that is suffering from and/or susceptible to a disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be an amount that provides a particular desired response in a significant number of subjects when administered to patients in need of such treatment, e.g., in at least about 25%, about 30%, about 40%, about 50%, about 60%, about 70%, about 80%, about 90%, about 95%, or more patients within a treated patient population. In some embodiments, reference to a therapeutically effective amount may be a reference to an amount sufficient to induce a desired effect as measured in one or more specific tissues (e.g., a tissue affected by the disease, disorder or condition) or fluids (e.g., blood, saliva, serum, sweat, tears, urine). Those of ordinary skill in the art will appreciate that, in some embodiments, a therapeutically effective amount of a particular agent or therapy may be formulated and/or administered in a single dose. In some embodiments, a therapeutically effective agent may be formulated and/or administered in a plurality of doses, for example, as part of a dosing regimen.

Tumor: As used herein, the term “tumor” refers to an abnormal growth of cells or tissue. In some embodiments, a tumor may comprise cells that are precancerous (e.g., benign), malignant, pre-metastatic, metastatic, and/or non-metastatic. In some embodiments, a tumor is associated with, or is a manifestation of, a cancer. In some embodiments, a tumor may be a disperse tumor or a liquid tumor. In some embodiments, a tumor may be a solid tumor.

Upstream and downstream: As used herein when describing RNA the term “upstream” refers to toward or close to the 5′ end of the RNA molecule and the term “downstream” refers to toward or close to the 3′ end” of the RNA molecule. As used herein when describing DNA, “upstream” is toward the 5′ end of the coding strand and “downstream” is toward the 3′ end of the coding strand. Because of the anti-parallel orientation of DNA, this means the 3′ end of the template strand is upstream and the 5′ end is downstream.

Variant: As used herein in the context of molecules, e.g., nucleic acids, proteins, or small molecules, the term “variant” refers to a molecule that shows significant structural identity with a reference molecule but differs structurally from the reference molecule, e.g., in the presence or absence or in the level of one or more chemical moieties as compared to the reference entity. In some embodiments, a variant also differs functionally from its reference molecule. In general, whether a particular molecule is properly considered to be a “variant” of a reference molecule is based on its degree of structural identity with the reference molecule. As will be appreciated by those skilled in the art, any biological or chemical reference molecule has certain characteristic structural elements. A variant, by definition, is a distinct molecule that shares one or more such characteristic structural elements but differs in at least one aspect from the reference molecule. To give but a few examples, a polypeptide may have a characteristic sequence element comprised of a plurality of amino acids having designated positions relative to one another in linear or three-dimensional space and/or contributing to a particular structural motif and/or biological function; a nucleic acid may have a characteristic sequence element comprised of a plurality of nucleotide residues having designated positions relative to on another in linear or three-dimensional space. In some embodiments, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some embodiments, a variant polypeptide or nucleic acid shows an overall sequence identity with a reference polypeptide or nucleic acid that is at least 85%, 86%, 87%, 88%, 89%, 90%, 91%, 92%, 93%, 94%, 95%, 96%, 97%, or 99%. In some embodiments, a variant polypeptide or nucleic acid does not share at least one characteristic sequence element with a reference polypeptide or nucleic acid. In some embodiments, a reference polypeptide or nucleic acid has one or more biological activities. In some embodiments, a variant polypeptide or nucleic acid shares one or more of the biological activities of the reference polypeptide or nucleic acid. In some embodiments, a variant polypeptide or nucleic acid lacks one or more of the biological activities of the reference polypeptide or nucleic acid. In some embodiments, a variant polypeptide or nucleic acid shows a reduced level of one or more biological activities as compared to the reference polypeptide or nucleic acid. In some embodiments, a polypeptide or nucleic acid of interest is considered to be a “variant” of a reference polypeptide or nucleic acid if it has an amino acid or nucleotide sequence that is identical to that of the reference but for a small number of sequence alterations at particular positions. Typically, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant polypeptide or nucleic acid comprises about 10, about 9, about 8, about 7, about 6, about 5, about 4, about 3, about 2, or about 1 substituted residues as compared to a reference. Often, a variant polypeptide or nucleic acid comprises a very small number (e.g., fewer than about 5, about 4, about 3, about 2, or about 1) number of substituted, inserted, or deleted, functional residues (i.e., residues that participate in a particular biological activity) relative to the reference. In some embodiments, a variant polypeptide or nucleic acid comprises not more than about 5, about 4, about 3, about 2, or about 1 addition or deletion, and, in some embodiments, comprises no additions or deletions, as compared to the reference. In some embodiments, a variant polypeptide or nucleic acid comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some embodiments, a reference polypeptide or nucleic acid is one found in nature. In some embodiments, a reference polypeptide or nucleic acid is a human polypeptide or nucleic acid.

### DETAILED DESCRIPTION OF CERTAIN EMBODIMENTS

### Cancer

The present disclosure provides, among other things, methods and compositions useful in the treatment of cancer, e.g., for the treatment of a tumor in a subject.

Cancer is among the leading causes of death worldwide; the number of new cancer cases diagnosed per year is expected to exceed 23 million by 2030. According to statistics released by the United States National Cancer Institute, in 2018, more than 1.7 million new cases of cancer were diagnosed in the United States, and more than 600 thousand people died from the disease.

The most common cancers, in descending order, are breast cancer, lung and bronchus cancer, prostate cancer, colon and rectum cancer, melanoma of the skin, bladder cancer, non-Hodgkin lymphoma, kidney and renal pelvis cancer, endometrial cancer, leukemia, pancreatic cancer, thyroid cancer, and liver cancer. More than 35% of men and women are expected to be diagnosed with cancer at some point during their lifetimes.

In some embodiments, a tumor or cancer suitable for treatment in accordance with the present disclosure includes, for example, Acute Lymphoblastic Leukemia (ALL), Acute Myeloid Leukemia (AML), Adrenal Cortex Cancer, Adrenocortical Carcinoma, AIDS-Related Cancer (e.g., Kaposi Sarcoma, AIDS-Related Lymphoma, Primary CNS Lymphoma), Anal Cancer, Appendix Cancer, Astrocytoma , Atypical Rhabdoid Tumor, Basal Cell Carcinoma, Bile Duct Cancer, Bladder Cancer, Bone Cancer , Brain Tumor, Breast Cancer, Bronchial Tumor, Burkitt Lymphoma, Carcinoid Tumor , Carcinoma, Cardiac (Heart) Tumor, Central Nervous System Tumor , Cervical Cancer, Cholangiocarcinoma, Chordoma, Chronic Lymphocytic Leukemia (CLL), Chronic Myelogenous Leukemia (CML), Chronic Myeloproliferative Neoplasm, Colorectal Cancer, Craniopharyngioma, Cutaneous T-Cell Lymphoma, Ductal Carcinoma In Situ (DCIS), Embryonal Tumor , Endometrial Cancer, Endometrial Sarcoma, Ependymoma, Esophageal, Esthesioneuroblastoma, Ewing Sarcoma, Extracranial Germ Cell Tumor, Extragonadal Germ Cell Tumor, Eye Cancer, Fallopian Tube Cancer, Gallbladder Cancer, Gastric (Stomach) Cancer, Gastrointestinal Carcinoid Tumor, Gastrointestinal Stromal Tumor (GIST), Germ Cell Tumor, Gestational Trophoblastic Disease, Glioma, Hairy Cell Leukemia, Head and Neck Cancer, Hepatocellular (Liver) Cancer, Hodgkin Lymphoma, Hypopharyngeal Cancer, Intraocular Melanoma, Islet Cell Tumor , Kaposi Sarcoma, Kidney Tumor, Langerhans Cell Histiocytosis , Laryngeal Cancer, Leukemia, Lip and Oral Cavity Cancer, Liver Cancer, Lung Cancer, Lymphoma, Male Breast Cancer, Malignant Fibrous Histiocytoma, Melanoma, Merkel Cell Carcinoma, Mesothelioma, Mouth Cancer, Multiple Endocrine Neoplasia Syndrome, Multiple Myeloma, Plasma Cell Neoplasm, Mycosis Fungoides, Myelodysplastic Syndrome , Myelodysplastic/Myeloproliferative Neoplasm , Nasal Cavity Cancer, Nasopharyngeal Cancer, Neuroblastoma, Non-Hodgkin Lymphoma, Non-Small Cell Lung Cancer, Oral Cancer, Oral Cavity Cancer, Oropharyngeal Cancer, Osteosarcoma, Ovarian Cancer, Pancreatic Cancer, Pancreatic Neuroendocrine Tumor (Islet Cell Tumor), Paraganglioma, Paranasal Sinus Cancer, Parathyroid Cancer, Penile Cancer, Pharyngeal Cancer, Pheochromocytoma, Pituitary Tumor, Pleuropulmonary Blastoma, Primary Central Nervous System (CNS) Lymphoma, Primary Peritoneal Cancer, Prostate Cancer, Rectal Cancer , Renal Cell (Kidney) Cancer, Retinoblastoma, Retinoblastoma, Rhabdomyosarcoma, Rhabdomyosarcoma, Salivary Gland Cancer, Sarcoma, Sézary Syndrome, Skin Cancer, Small Intestine Cancer, Soft Tissue Sarcoma, Squamous Cell Carcinoma, Squamous Neck Cancer, Stomach (Gastric) Cancer, T-Cell Lymphoma, Testicular Cancer, Testicular Cancer, Throat Cancer, Thymic Carcinoma, Thymoma, Thyroid Cancer, Urethral Cancer, Uterine Sarcoma, Uterine Sarcoma, Vaginal Cancer, Vascular Tumor, Vulvar Cancer, Waldenström Macroglobulinemia, Wilms' Tumor. In some preferred embodiments, a tumor or cancer suitable for treatment in accordance with the present disclosure comprises cancers with high frequency of p53 mutation or inactivation, including lung cancer (both non-small cell lung cancer and small cell lung cancer), colon cancer, pancreatic cancer, head and neck cancer, esophageal cancer, ovarian cancer (e.g. high-grade serous ovarian cancer), bladder cancer, liver cancer, gastric cancer, melanoma, AML (e.g. therapy related AML, complex Karyotype AML, AML with 17p deletion), chronic myeloid leukemia, and Burkitt's lymphoma.

### Ribosomes

A ribosome is the center of protein synthesis. Ribosomes synthesize proteins by linking individual amino acids together as directed by a nucleic acid code. The eukaryotic ribosome is a complex macromolecular machine made of 4 rRNA species and 80 ribosomal proteins (RPs). The mature ribosome is composed of 2 subunits, the small 40S ribosomal subunit containing the 18S IRNA and 33 RPs and the large 60S ribosomal subunit containing the 28S, 5.8S, and 5S IRNAs and 47 RPs. rRNA is heavily modified including features such as base methylation, pseudouridylation, and ribose methylation at 2′-hydroxyl (2′-O-methylation). The most abundant rRNA modifications are isomerisation of uridine into pseudouridine by pseudouridine synthases and H/ACA box small nucleolar RNAs (snoRNAs) and 2′-O-methylation of the ribose, performed by the methyltransferase Fibrillarin (FBL).

Typically, a ribosome will “read” instructions in an RNA code; in some embodiments, the nucleic acid containing the code is an mRNA. In some embodiments, structural features of the ribosome interact with the string of amino acids, the nascent polypeptide, being generated by the ribosome activity. In some, embodiments the ribosome can affect folding of the nascent polypeptide.

### Oncoselective Translation

### Oncogenic Ribosomes

The present disclosure appreciates that studies have increasingly revealed alterations in ribosome structure and function that are associated with tumor development and/or progression. See, for example, Bastide and David Oncogenesis 2018 Apr. 7(4):34. Oncogenic ribosomes have a drastically altered translational landscape (“translatome”). In addition to more effectively translating various oncogenes, cancer ribosomes have been shown to be characterized by low translation fidelity and/or altered or increased stop codon read-through.

A variety of mechanisms have been described that may contribute to the altered function of oncogenic ribosomes. These include alterations in ribosome biogenesis, mutations in ribosomal protein genes, alterations in expression of ribosomal proteins, alterations in expression of rRNA, and/or alterations in the modification of rRNA. See, for example, Bastide and David Oncogenesis. 2018 Apr; 7(4): 34. Alterations in rRNA 2′-O-methylation patterns are also involved in cancer evolution. In some cancers, p53 inactivation triggers FBL overexpression and subsequent changes in rRNA methylation landscape (Marcel et al. Cancer Cell. 2013;24:318-330). Such p53 inactivation (and/or FBL overexpression and/or changes in rRNA methylation) result(s) in impaired translational fidelity and increased translation of IRES-containing mRNAs. The gene encoding p53 protein, TP53 is the most commonly mutated tumor suppressor gene, Along with IRNA modifications, it is also closely connected with ribosome regulation through changes in ribosomal proteins. Ribosomal protein gene haploinsufficiency is found in about 43% of all cancers (Ajore et al., EMBO Mol Med. 2017;9(4):498-507). In healthy cells, loss of both copies of any essential ribosomal protein gene is lethal. However, when a single copy of a ribosomal protein gene is lost, the stoichiometry of ribosomal proteins is altered and ribosomal proteins RPL5 and RPL11 have higher free (unbound) forms, which together with 5S rRNA, bind to MDM2 and stabilize p53 to stimulate growth arrest or apoptosis. This p53 mediated control mechanism in healthy cells is termed “impaired ribosome biogenesis checkpoint (Gentilella et al. Mol Cell. 2017;67(1):55-70.e4).” In addition to TP53, retinoblastoma (RB1) gene, another commonly mutated tumor suppressor gene, is also involved in ribosome regulation, suppressing translational read-through in MYC oncogene-transformed senescent human cells (del Toro et al. BioRxiv. 2019; 10.1101/788380).

The present disclosure appreciates that oncoselective read-through can be harnessed as a powerful strategy for treatment of cancer. The present disclosure builds upon extensive work in the field of nucleic acid therapeutics (and particularly including RNA, such as mRNA therapeutics), among other things by providing technologies that ensure expression of a payload included in and/or encoded by such a nucleic acid is selectively or specifically expressed in tumor cells (relative to non-tumor cells).

By providing true oncoselective or oncospecific expression, the present disclosure reduces or obviates a need to develop and/or utilize targeted (e.g., oncoselective) delivery strategies that may be required in contexts where oncoselective or oncospecific payload expression cannot be achieved. Of course, those skilled in the art, reading the present disclosure, will appreciate that any available such oncoselective delivery technology may, in some embodiments, be desirably combined with provided technologies; it is simply not required.

Alternatively or additionally, by providing true oncoselective or oncospecific expression, the present disclosure creates an option to utilize payloads that might be inappropriate or undesirable without such a high degree of selectivity. For example, as discussed herein, cytotoxic payloads (e.g., such as toxins, and pro-necroptotic, pro-pyroptotic, and pro-apoptotic proteins) might have unacceptable side effect and/or toxicology profiles when utilized with technologies that cannot ensure oncoselectivity to the extent described herein.

### Oncoselective Translation Sequence Elements

### Read-Through Motifs

Among other things, the present disclosure encompasses the recognition that different ribosomes (e.g., ribosomes in tumor cells—e.g., oncogenic ribosomes—vs ribosomes in non-tumor cells—e.g., non-oncogenic ribosomes) have different processivity and/or read-through properties (e.g., different responses to pause structures and/or stop codons that impact processivity therethrough). In some embodiments, oncogenic ribosomes have frame shifts relative to non-oncogenic ribosomes. In some embodiments, frame shifts by oncogenic ribosomes can result in expression of payload sequences described herein.

In some embodiments, oncogenic ribosomes read-through, or process through, a canonical stop codon. In some embodiments, read-through of a stop codon by an oncogenic ribosome results in translation of a stop codon into an amino acid incorporated into a nascent polypeptide. In some embodiments, read-through of a stop codon by an oncogenic ribosome results in translation of some portion or all of the downstream (3′UTR) sequences following that stop codon.

Without wishing to be bound by any particular theory, the present disclosure observes that ribosome read-through of stop codons can be caused by interactions between the 18s rRNA and an RNA (e.g., an mRNA) bound by the ribosome. For example, helices of the IRNA may interact with mRNA sequences. See Namy et al. EMBO Rep. 2001 Sep 15; 2(9): 787-793 describing interactions of helix 17 of rRNA in S. cervisae with mRNA bound by the ribosome that leads to stop codon read-through. The present disclosure recognizes, among other things, that human rRNA helix 37 can interact with sequences of mRNA that contribute to stop codon read-through.

Alternatively or additionally, oncoselective ribosome stop codon read-through can be induced and/or enhanced by including one or more particular structural features in a translatable nucleic acid (e.g., an RNA such as an mRNA). In some embodiments, one or more primary structure features of a translatable nucleic acid (e.g., an RNA such as an mRNA) can be used to induce and/or enhance oncoselective stop codon read-through. Alternatively or additionally, in some embodiments, one or more secondary and/or tertiary structure features (e.g. stem loop, bulge loop, kissing loop, pseudoknots, or branch loop) of a translatable nucleic acid (e.g., an RNA such as an mRNA) can be used to induce and/or enhance oncoselective stop codon read-through. In some embodiments, a structural feature capable of inducing and/or enhancing stop codon read-through is within the first 50, 60, 70, 80, 90, 100, 110, or 120 nucleotides of the downstream flanking sequence. In some further embodiments, portions of a structural feature capable of inducing and/or enhancing stop codon read-through is comprised by the first 16 nucleotides of the downstream flanking sequence.

In some embodiments, a structural feature capable of inducing and/or enhancing stop codon read-through comprises 10, 20, 30, 40, 50 or more base paired nucleotides within the first 10, 20, 30, 40, 50, 60 or more nucleotides of the downstream flanking sequence.

In accordance with some embodiments of the present disclosure, stop codon read-through can be induced and/or enhanced through use of oncoselective read-through motifs as described herein.

Alternatively or additionally, in some embodiments, inclusion of one or more regions of high G-C content can be used to induce oncospecific stop codon read-through. For example, in some embodiments, high G-C content in the 3′UTR of a translatable nucleic acid (e.g., of an RNA such as an mRNA) can be used to induce and/or enhance oncospecific stop codon read through. In some embodiments, high G-C content in the nucleotides preceding a stop codon can be used to induce and/or enhance oncospecific stop codon read-through of that stop codon. In some embodiments, high G-C content in the 60 nucleotides preceding a stop codon can be used to induce and/or enhance oncospecific stop codon read-through of that stop codon. In some embodiments, high G-C content in 50 nucleotides following a stop codon can be used to induce and/or enhance oncospecific stop codon readthrough of that stop codon. In some embodiments, high G-C content in the first 120 nucleotides after a stop codon (i.e., in the 3′UTR) can be used to induce and/or enhance oncospecific stop codon readthrough of that stop codon. In some embodiments, high G-C content means a log-odds of binomial probability of 4 or greater relative to a non-readthrough transcript. In some embodiments, a readthrough motif comprises GC content of more than 42%, more than 48%, preferably more than 54% in the downstream flanking sequence.

In some embodiments, the readthrough motif comprises the amino acid sequence VNNNNNNMNNMWK (SEQ ID NO. 24), NNNVWNNKGHHNH (SEQ ID NO. 25), DVHVNNNCWNNNB (SEQ ID NO. 26), MWBNNNNNNNNNN (SEQ ID NO. 27), WGNNSNHNHDNNN(SEQ ID NO. 28), VNNNNNNMNNMWK(SEQ ID NO. 29) or VMNNWNKNNNNNN (SEQ ID NO. 30), wherein V stands for A, C or G, M stands for A or C, W stands for A or T/U, K stands for G or T/U, H stands for A, C or T/U, D stands for A,G or T/U, B stands for C, G or T/U, S stands for G or C, N stands for any nucleotide, within the region that spans the readthrough stop codon and the first 14 nucleotides of the downstream flanking sequence.

The present disclosure further provides an insight that inclusion of a codon resulting in introduction of proline to the nascent polypeptide can induce kinking of the nascent polypeptide, and that such kinking can be used to induce and/or enhance oncoselective stop codon read-through. Thus, in some embodiments, oncoselective stop-codon read through can be induced and/or enhanced by inclusion of one or more proline-encoding codons in a translatable nucleic acid, as an alternative to or in addition to one or more of the other strategies described herein for inducing and/or enhancing oncoselective stop codon read-through.

In some embodiments, a stem loop in the mRNA can induce and/or enhance stop codon read-through. In some embodiments, a stem loop inducing and/or enhancing stop codon readthrough is within approximately 20, 40, 60, 80 or 120 nucleotides of the stop codon. In some embodiments, a stem loop inducing and/or enhancing stop codon read-through is in the coding sequence just prior to the stop codon. In some embodiments, a stem loop inducing and/or enhancing stop codon read-through is in the 3′UTR. In some embodiments, a stem loop inducing and/or enhancing stop codon read-through is in the region spanning the coding region and 3′UTR boundary. In some embodiments, a bulge loop or a pseudoknot in the mRNA can induce and/or enhance stop codon read-through. In some embodiments, nucleic acid structures inducing and/or enhancing stop codon read-through have a low Gibbs free energy relative to nucleic acid structures that do not result in read-through. In some embodiments, the first 25, 50, or 75 nucleotides of the 3′UTR of a nucleic acid inducing stop codon read-through have a delta G of 5 kcal/mole; 10 kcal/mole; 15 kcal/mole; 20 kcal/mole; 25 kcal/mole; 30 kcal/mole lower than non-cancer stop codon read-through counterparts. In some embodiments, the first 25, 50, or 75 nucleotides of the 3′UTR of a nucleic acid inducing stop codon read-through have a delta G in the range of 5 kcal/mole to 20 kcal/mole; 5 kcal/mole to 10 kcal/mole; or 10 kcal/mole to 20 kcal/mole; 25 kcal/mole; 30 kcal/mole lower than non-cancer stop codon read-through counterparts.

In some embodiments, aminoglycosides (e.g., gentamicin) and macrolides (e.g. erythromycin) can induce stop codon read-through. Without wishing to be bound by any theory, aminoglycosides can induce stop codon read-through by binding 18s rRNA and macrolides can induce stop codon read-through by binding the peptide channel within large ribosomal subunit. In some embodiments, aminoglycosides and macrolides can induce stop codon read-through in healthy (normal) cells. In some embodiments, subjects treated with aminoglycosides or macrolides should not be treated with a nucleic acid comprising a stop codon read-through motif.

In some embodiments, the present disclosure encompasses the recognition that an oncoselective translation sequence element can be oncospecific and result in translation and payload expression only in cancer cells (i.e., no detectable expression in non-cancer cells). Alternatively or additionally, in some embodiments, an oncoselective translation sequence element is translated 2, 5, 10, 15, 20, 30 or more -fold higher in cancer cell(s) as compared with appropriately comparable non-cancer cells.

In some embodiments, an oncoselective translation sequence element can comprise an internal ribosome entry segment/site (IRES). In some embodiments, an oncogenic ribosome, or RNA binding protein, preferentially binds an IRES in an oncoselective translation sequence element. In some embodiments, an oncoselective translation sequence element can be bound by or direct the binding of translation initiating RNA binding proteins (RBPs). In some embodiments, an oncoselective translation sequence element can comprise and IRES and be bound by or direct the binding of RBPs.

### Assessing Read-Through

The present disclosure provides a variety of insights relating to assessment (e.g., identification and/or characterization) of stop-codon readthrough that are useful in the identification and/or characterization of oncoselective translation sequence elements as described herein.

For example, as described in the Exemplification herein, the present disclosure identifies the source of a problem with certain common approaches to assessing stop codon read-through. Among other things, the present disclosure appreciates that many prior approaches have relied on analysis of either ribosome occupancy (e.g., via ribosome profiling and/or RNA Seq studies) or polypeptide production (e.g., vis mass spectrometry). The present disclosure provides an insight that such approaches can give false-positive and/or false-negative results due to biases inherent in the technologies but not always appreciated. In some embodiments, the present disclosure teaches that stop codon read through is desirably assessed through use of a combination of technologies that independently assess (i) ribosome occupancy or location; and (ii) production of a read-through polypeptide.

The present disclosure further provides an insight that many prior approaches to assessing stop-codon read-through have compared observed levels or features (whether determined by ribosome profiling, RNA Seq, mass spectrometry, or one or more other technologies, or any combination thereof) with a “reference” that itself has one or more cancer-related features and therefore does not provide a true comparison with a “non-cancer” reference as described herein.

For example, the present disclosure recognizes that many cell lines include one or more cancer-related features that reduce their usefulness as a reference for assessing oncoselective stop-codon readthrough as described herein. HEK293 cells, for example, are preferably not used as a “non-cancer” reference for assessing oncoselective stop-codon readthrough in many embodiments of the present disclosure, as these cells may contain one or more viral gene insertions, such as Adenoviral E1B gene, which deactivate p53 and transform the cells into an immortal and tumorigenic cell line and may impact their performance in such assessments and distort or destroy analyses attempting to identify oncoselectivity.

### Nucleic Acids

Among other things, the present disclosure provides nucleic acids that participate in and/or are otherwise related to oncoselective translation as described herein. In some embodiments the present disclosure provides nucleic acids that are or include or deliver a translatable nucleic acid comprising an oncoselective read-through motif. In some embodiments, the present disclosure provides nucleic acids that are or include or deliver a translatable nucleic acid encoding a payload of interest and including an oncoselective translation sequence element as described herein.

In some embodiments, a provided nucleic acid may be or comprise DNA (e.g., single or double-stranded DNA), e.g., that, when introduced into a cell, is transcribed, or generates a template strand that is transcribed) to produce a translatable nucleic acid (e.g., an RNA such as an mRNA) as described herein. In some embodiments, a provided nucleic acid may be or comprise RNA (e.g., mRNA), which may be or comprise (or may be or comprise the complement of) a translatable nucleic acid described herein (e.g., may be or comprise a coding sequence and an oncoselective translation sequence element(s)).

In some embodiments, a provided nucleic acid is or comprises DNA or RNA or both. In some embodiments, a provided nucleic acid is chemically modified relative to naturally-occurring DNA and/or RNA. In some embodiments, a provided nucleic acid is not modified with pseudouridine.

In some embodiments, a provided nucleic acid is a translatable nucleic acid as described herein. In some embodiments, a provided nucleic acid is expressible (e.g., can be transcribed to express) to produce a translatable nucleic acid as described herein. In some embodiments, a provided nucleic acid is a complement of a translatable nucleic acid as described herein, or of a nucleic acid that is expressible to produce such a translatable nucleic acid (or its complement).

Thus, in some embodiments, the present disclosure builds upon and enhances recent developments in the field of RNA (e.g., mRNA) therapeutics. Several groups have done important work developing technologies for, for example, improving RNA production and/or stability; providing encapsulating or other systems to facilitate RNA administration and/or delivery to mammalian (e.g., human) subjects; etc. Recent work by companies such as BioNTech AG, Cure Vac AG, Ethris AG, Moderna Therapeutics, Translate Bio, Inc., and others have led to development of several clinical candidates, and, recently, the first RNA therapeutic approved by the US Food and Drug Administration; those skilled in the art will appreciate that any or all of the available technologies for production, stability, administration, etc of RNA therapeutics may be applicable to and/or utilized with those embodiments of the present disclosure that administer a translatable RNA to mammalian (e.g., human) subjects.

Analogously, the present disclosure builds upon and enhances various developments in the field of gene therapy, e.g., involving development of DNA and/or RNA vectors that can deliver translatable nucleic acids to cells in mammalian (e.g., human) subjects. Recent work on oncolytic viruses have demonstrated efficient gene delivery and cell killing in various malignancies (Raman et al., Immunotherapy. 2019 Jun; 11(8): 705-723; Mahalingam et al., Cancers (Basel). 2018 May 25;10(6)). In addition, groups working on self-amplifying mRNA replicons have demonstrated efficient local delivery and improved pharmacokinetic profile with prolonged protein expression (Avogadri et al, Cancer Immunol Res. 2014 May;2(5):448-58; Huysmans et al., 2019 bioRxiv 10.1101/528612). In some embodiments of the present disclosure, a provided nucleic acid comprises an oncolytic virus particle or an oncolytic DNA or RNA or a self-amplifying mRNA formulated in polymer or lipid nanoparticle.

In some embodiments, a provided nucleic acid is engineered to show low or reduced (relative to an appropriate reference) immunogenicity when introduced, produced, and/or expressed in a subject. Those skilled in the art are aware of certain sequence elements and/or chemical modifications that can increase or decrease immunogenicity of a nucleic acid that contains them as compared with one that does not. In many embodiments, provided nucleic acids are engineered so that those that are or will be introduced, produced, and/or expressed in a subject are characterized by low expected or observed immunogenicity. For example, provided mRNAs can be engineered by increasing GC content (Thess et al., 2015, Mol Ther. 23:1456-64) or decreasing U content (Kariko & Sahin, 2017, WIPO Patent App No: WO 2017/036889 A1; Vaidyanathan, et al., 2018. 12: 530-542). The provided mRNAs can contain modified by incorporation of non-canonical nucleotides, such as pseudouridine, N1-methyl-pseudouridine, methoxy-uridine, and 2-thiouridine into mRNA (Kariko, 2005, Immunity. 23:165-75; Kariko, 2008, Mol Ther. 16:1833-40; Kormann et al., 2011, Nat Biotechnol. 29:154-157; Andries et al., 2015, J Control Release. 217:337-344).

Alternatively or additionally, in some embodiments, a provided nucleic acid that includes or encodes a translatable payload is engineered so that the payload, when introduced and/or produced in a subject, shows relatively low immunogenicity. For example, in some embodiments, immunogenic epitope(s) may have been defined for a particular payload, and a less-immunogenic variant (e.g., having a sequence alteration within, or that otherwise impacts immunogenicity of such as by altering a pattern of post-translational modification, one or more such immunogenic epitope(s)) may be utilized in accordance with the present disclosure.

### Coding Sequence

As described herein, the present disclosure relates particularly to translatable nucleic acids that comprise a coding sequence (e.g., a payload coding sequence) and an oncoselective translation sequence element.

Those of ordinary skill in the art, reading the present disclosure, will appreciate that a wide variety of useful payload sequences are known and can be utilized in accordance with the teachings herein. In some embodiments, the payload is a gene product (e.g., a polypeptide) that, when expressed in cancer cells, reduces their ability to survive and/or to proliferate within a subject.

In some embodiments, a payload sequence may be toxic to cells and/or may generate (e.g., enzymatically) a toxic agent.

In some embodiments, a payload sequence may render cells more susceptible to immunological attack and/or clearance. For example, in some such embodiments, a payload sequence may be or comprise an antigen, antibody, antibody fragment, or their chimeric versions fused to a transmembrane protein and/or an intracellular signaling molecule (e.g. ITAM or costimulatory molecule endodomains) that is particularly attractive to a subject's immune system and/or to an immunological therapy (e.g., CAR-T or CAR-NK cells, proliferated T-cells, etc) that has been or will be administered to the subject. Alternatively or additionally, in some such embodiments, a payload sequence may be or comprise an agent that relieves or inhibits an immunological checkpoint.

As noted herein, one feature of the provided disclosure is that it achieves an extent of oncoselectivity such that payloads that would be unacceptable and/or inadvisable without such oncorestricted expression may be effectively utilized.

In some embodiments, a payload sequence for use in accordance with the present disclosure is selectively active in cancer cells and/or under particular circumstances (e.g., in the presence of a separate agent). However, in some embodiments, particularly in light of the degree of oncoselectivity provided by the present disclosure, in some embodiments, a payload comprises a protein that is constitutively active and/does not require post-translational modifications such as cleavage or phosphorylation.

In some embodiments, a payload is not secreted from a cell in which it is produced (e.g., by translation). In some other embodiments, a payload is a protein that is secreted into the tumor microenvironment.

In some embodiments, a polypeptide payload may be or comprise an antibody, a cell surface protein (e.g., that is or comprises an antigen or epitope targeted by endogenous or administered immune cells—such as T cells, NK cells, etc), an enzyme, a genetic modification protein, a suicide protein, a toxin, a viral replication protein, a viral surface antigen, etc. In some embodiments, a polypeptide payload may be or comprise a biologic agent approved for treatment of cancer.

In some embodiments a linker may be present between an oncoselective translation sequence element and a payload sequence. In some embodiments, a linker comprises 2A linker. In some embodiments, a linker comprises a PT2A linker. In some embodiments, a linker comprises a F2Am linker.

### Antibody Agents

Several antibody therapeutics useful in the treatment of cancer are known in the art. Recent developments in the mRNA therapeutic field indicate that delivery of a translatable nucleic acid encoding an antibody agent of interest can be a viable and effective strategy for administering antibody therapeutics (see, for example, Van Hocke & Roose, J. Translational Med. 17:54,Feb. 22, 2019). Those skilled in the art, reading the present disclosure, will appreciate that its teachings are applicable to therapeutic antibody agents; in some embodiments, a translatable nucleic acid as described herein encodes a polypeptide that is or is a component of a therapeutic antibody agent. In some embodiments of the present disclosure, such agents may be antibody agents against receptor tyrosine kinases (e.g. EGFR, Her2, CD20, FGFR) or pro-angiogenic factors (e.g. VEGF, VEGFR, PDGF, PDGFR). In some other embodiments, the payload may be an antibody agent (e.g., asingle chain variable fragment (scFv), nanobody, or bispecific antibody), fusion protein, or a synthetic polypeptide.

### Immune Checkpoint Inhibitors and Modulators

Immune checkpoint are the regulators of immune system. They play a significant role in the immune evasion and escape of human tumors. Their modulators have shown significant efficacy in the cancer therapeutics field (see Wei et al., Cancer Discov. 2018. 10.1158/2159-8290). When secreted from tumors the intratumoral concentrations of such immune modulators are higher and their systemic concentrations are lower. This improved pharmacokinetic profile can boost the efficacy and lower the toxicity associated with these agents. In some embodiments, a payload may be or comprises an immune checkpoint inhibitor, i.e. an antagonist antibody agent against immune checkpoint proteins, e.g. anti-PD1, anti-PDL1, anti-CTLA-4, anti-TIM3, anti-BTLA, anti-VISTA, anti-LAG-3, anti-TIGIT, anti-CD39, anti-SIRP-α. In some other embodiments, a payload may be or comprises an agonist antibody against CD-28, OX40, GITR, CD137, CD27, HVEM, or CD27. In some other embodiments, the payload may be a costimulatory molecule such as CD80, CD86, and OX40L.

### Cytokines

Cytokines have critical roles in regulation of immune cells. IL-2 and IFN-alpha were the first two immunotherapy cytokines that were FDA approved for the treatment of metastatic melanoma and renal cell carcinoma (high dose, bolus Il-2) and Stage III melanoma (IFN-alpha) (Lee and Margolin, Cancers (Basel). 2011 Dec; 3(4): 3856-3893). However, their clinical use is limited by systemic toxicity issues (Rosenberg, J Immunol, 2014, 192 (12) 5451-5458). Those skilled in the art will appreciate that onco-selective production and secretion of cytokines can greatly improve their therapeutic window. In some embodiments, a payload for use in accordance with the present disclosure may be IL-2, IL-2 superkine/mutein, IL-12, IL15, IL15, IL15R-alpha fusion, Il-23, IL-36, TNF-alpha, IFN-alpha, IFN-gamma, FLT3 ligand, CCL4, RANTES, GM-CSF, or engineered variants or fusions thereof.

### Modulators of Tumor Microenvironment

In human cancer, the tumor microenvironment is frequently altered to prevent or suppress anti-tumor immune response (Binnewies et al., Nature Medicine, 24, 541-550, 2018; Valkenburg et al., Nature Reviews Clinical Oncology, 15, 366-381, 2018). There are various modulators of tumor microenvironment that alter the extracellular matrix to enhance immune cell infiltration or that inflame the milieu to turn cold tumors into hot tumors. Some of these modulators have shown signs of efficacy in the preclinical models. However, some others were not dropped during preclinical or clinical development due to systemic toxicity issues (see, for example, Ramanathan et al, Journal of Clinical Oncology, Jan 18-20, 2018 36.4_suppl.208). Present disclosure teach ways, to those skilled in the art, that would allow for the local secretion of such immune modulators, which can enhance intratumoral activity while minimizing systemic effects. In some embodiments, a payload may be a protein such as a kynureninase, adenosine deaminase (ADA2) and 15-hydroxyprostaglandin dehydrogenase (15-PGDH). In some other embodiments, a payload may be an enzyme, such as hyaluronidase and colleganase, which degrades the extracellular matrix and alters the tumor stroma.

### Cell Surface Antigens

Those skilled in the art are aware of various therapeutic technologies that have been developed for treating cancer by immunologically targeting antigens or epitopes expressed on surfaces of tumor cells. In some embodiments, a payload encoded by a translatable nucleic acid for use in accordance with the present disclosure encodes such an antigen or epitope, that may be immunologically targeted by a subject's immune system and/or by immune therapy (e.g., cell therapy such as CAR-T or CAR-NK therapy, or adoptive immunotherapy) administered to the subject. In some embodiments, such a cell surface antigen or epitope may be or comprise an antigen or epitope already expressed by relevant cancer cells; without wishing to be bound by any particular theory, the present disclosure proposes that increased expression of such an antigen or epitope may facilitate its targeting. In some embodiments, such an antigen or epitope may be one not already expressed by relevant tumor cells; in some such embodiments, it may be selected to permit targeting by an existing immune response or therapy.

### Genetic Modification Proteins

Those skilled in the art, reading the present disclosure, will be aware of the relevance of its teachings to genetic modification enzymes and their use, for example, to modify or destroy one or more aspects of a cancer cells' genome, transcriptome, etc.

For example, in some embodiments, a payload encoded by a translatable nucleic acid as described herein may be or comprise a genetic modification protein (e.g., that is or comprises a nuclease). In some embodiments, a a genetic modification enzyme may be or comprise a transcription activator-like effector nuclease (TALEN), a zinc finger nuclease (ZFN), one or more components of a CRISPR based gene modification system (e.g., a Cas enzyme).

In some embodiments, a genetic modification protein (e.g., a nuclease) that targets sequences found preferentially or only in relevant cancer cells. However, those skilled in the art, reading the present disclosure, will appreciate that the degree of oncoselectivity in achieves permits use of genetic modification proteins that target sequences that are not particularly specific to cancer cells, as the genetic modification protein itself will be preferentially expressed only in those cells.

### Suicide Proteins

Those skilled in the art will be aware of various proteins commonly referred to as “suicide proteins” (encoded by “suicide genes”) and will appreciate that, in some embodiments, a payload sequence included in a translatable nucleic acid as described herein is or comprises a suicide protein.

In some embodiments, a suicide protein is a protein that induces cell death. In some embodiments, a suicide protein is a protein that induces immunogenic cell death, such as necroptosis, pyroptosis or ferroptosis. The present disclosure provides an insight that certain suicide proteins that induce necroptosis may be particularly advantageous for use in accordance with the present disclosure. For example, the present disclosure observes that necroptosis can induce and/or promote an adaptive immune response. Without wishing to be bound by any particular theory, the present disclosure observes that necroptosis involves immune ligands including Fas, TNF, and LPS leading to activation of RIPK. Dhuriya and Sharma J Neuroinflammation. 2018 Jul 6;15(1): 199; Linkermann and Green N Engl J Med. 2014 Jan 30; 370(5): 455-465. The present disclosure teaches that use of a necroptotic suicide protein, which may induce and/or promote an adaptive immune response, may facilitate inhibition, destruction and/or removal of tumor cells. In some embodiments, a suicide protein induces apoptosis; in some such embodiments, a suicide protein is p53, or is a protein involved in a p53-mediated apoptosis pathway (e.g. PUMA, BIM, BAX, BAK, tBID, CASPASE-3, CASPASE-7, CASPASE-8, CASPASE-9).

In some embodiments, a suicide protein is or comprises a protein that renders cells expressing it more susceptible to killing by a separate agent. To give but one example, those skilled in the art are aware of certain viral and/or bacterial enzymes that are not naturally found in mammals and that convert a substance that may be harmless to cells that do not express the enzyme(s) into a toxin. In some embodiments, such a suicide protein is or comprises an enzyme that converts an otherwise inactive agent (e.g., drug) into a toxic antimetabolite, e.g., that inhibits nucleic acid synthesis. In some such embodiments, a suicide protein is a thymidine kinase, wherein the payload sequence encoding thymidine kinase is co-administered with or administered before ganciclovir or valacyclovir treatment.

In some embodiments, a suicide protein payload for use in accordance with the present disclosure is Mixed Lineage Kinase Domain Like Pseudokinase (MLKL), Receptor-interacting serine/threonine-protein kinase 3 (RIPK3), Receptor-interacting serine/threonine-protein kinase 1 (RIPK1), Fas-associated protein with death domain (FADD), or gasdermin D (GSDMD), cysteine-aspartic proteases, cysteine aspartases or cysteine-dependent aspartate-directed proteases (CASPASE-1 or CASP-1), CASPASE-4, CASPASE-5, CASPASE-12, PYCARD/ASC (PYD and CARD domain containing/Fas-associated protein with death domain) or variants thereof.

### Toxins

In some embodiments, a payload for use in accordance with the present disclosure may be or include a toxin protein. Those skilled in the art will be aware of a variety of toxin proteins that may be useful to kill cancer cells. As noted herein, it is one feature of the present disclosure that the degree of oncoselectivity achieved is such that even very potent payloads may be utilized notwithstanding that such payloads might have significant deleterious effects if expressed in non-cancer cells. In some embodiments, a payload is a toxin that is not secreted from a cancer cell.

In some embodiments, a toxin may be or comprise a bacterial toxin. In some embodiments, a toxin may be or comprise a toxin produced by a venomous animal (see, for example, Kozlov et al Rec Pat DNA Gene Sequ1:200, 2007). In some embodiments, a toxin may be or comprise a plant toxin.

In some embodiments, a toxin that may be utilized as a payload in accordance with the present disclosure may be or comprise a phospholipase or a lecithinase. In some embodiments, a useful toxin may be or comprise a lethal toxin. In some embodiments, a useful toxin may be or comprise an exotoxin. In some embodiments, a useful toxin may be or comprise a pore-forming toxin. In some embodiments, a useful toxin may be or comprise a pyrogenic exotoxin.

In some embodiments, a toxin that may be utilized as a payload is one found in (or derived from) a bacterium that is a bacillus (e.g., Bacillus anthracis), bortadella (e.g., Bortadella pertussis), clostridium (Clostridium botulinum), corynebacterium (e.g., Corynebacterium diphtheriae), eschericia (e.g., Eschericia coli), listeria (e.g., Listeria monocytogenes), pseudomonas (pseudomonas aeruginosa), staphylococcus (e.g., Staphylocococus aureus), streptococcus, shigella (e.g. shigella dysenteriae),

In some embodiments, a toxin may be or comprise cholera toxin (e.g., A-5B), diphtheria toxin (e.g., A/B), pertussis toxin (e.g., A-5B), E. coli heat-labile toxin LT (e.g., A-5B), shiga toxin (e.g., A-5B), pseudomonas exotoxin (e.g., A/B), botulinum toxin (e.g., A/B), tetanus toxin (e.g., A/B), anthrax toxin (e.g., lethal factor [LF]), staphylococcus aureaus exfoliatin B.

In some embodiments, a toxin may be or comprise perfringiolysin (e.g., from clostridium perfringens), hemolysin (e.g., from eschericia coli), listeriolysin (e.g., from listeria monocytogenes), anthrax EF (e.g., from bacillys anthracis), alpha toxin (e.g., from staphylococcus aureaus), pneumolysin (e.g., from streptococcus pneumoniae), streptolysin PO (e.g., from streptococcus pyogenes), leucocidin (e.g., from staphylococcus aureus).

In some embodiments, a toxin may be a component of an exotoxin (e.g. Lethal Factor of anthrax toxin), that is, on its own, not capable of being internalized into mammalian cells.

In some embodiments, a toxin may be or comprise ricin or an amanitin. In some embodiments, a toxin may be or comprise alpha- amanitin.

### Inducible or Repressible Proteins

Recent advances in genetic engineering and synthetic biology allow for proteins that are inducible or repressible via small molecule modulators. In some embodiments, a repressible protein can be fused to a Ligand-Induced Degradation (LID) domain, which results in the proteolytic cleavage of the protein upon treatment with the small molecule Shield-1. In some other embodiments, an inducible protein may be inducible Caspase-9, which is activated by the small molecule rimiducid by dimerization. The activated Caspase-9 leads to rapid apoptosis of cells. In some other embodiments, the induction or repression may be achieved via other degradation domains (e.g. dihydrofolate reductase based destabilization domain) or dimerization domains (e.g. FKBP-FRB) and/or other small molecules (e.g. doxycycline, rapamycin, trimethoprim). In some embodiments, a payload for use in accordance with the present disclosure may be or include an inducible or repressible protein.

### Viral Proteins

Those skilled in the art are aware of a variety of viruses that produce proteins useful as payloads as described herein. In some embodiments, a payload may be or comprise a viral protein. In some embodiments, a payload may be LMP1 protein of Epstein-Barr virus. In some embodiments a payload may be or comprise an oncolytic virus protein.

In some embodiments, a payload may be or comprise a viral replication protein. In some embodiments, the viral replication protein is a protein needed for the viral replication cycle. In some embodiments, the viral replication protein is an enzyme. In some embodiments, the viral replication protein is a protease, a polymerase, or a transcriptase.

### Production

Those skilled in the art, reading the present disclosure, will appreciate that a variety of technologies are available that can usefully be employed to produce a translatable nucleic acid as described herein. In some embodiments, such production may be ex vivo (i.e., outside of a subject in need of cancer treatment as described herein); in some embodiments, such production may be in vivo.

In some embodiments, a translatable nucleic acid may be produced, wholly or partially, by chemical synthesis and/or chemical modification (e.g., capping)

In some embodiments, a translatable nucleic acid may be produced, wholly or partially, by copying (e.g., via replication or transcription) of a template nucleic acid. In some embodiments, such copying may be ex vivo; in some embodiments, it may be in vivo.

### Delivery

Those skilled in the art, reading the present disclosure, will appreciate that a variety of technologies are available to achieve delivery of a translatable nucleic acid to (at least) cancer cells in accordance with the present disclosure, and furthermore will appreciate that some modes of delivery involve administration of a composition comprising the translatable nucleic acid (e.g., mRNA), and some modes of delivery involve administration of a composition from which the translatable nucleic acid is generated after administration (e.g., via administration of a vector that encodes or templates the translatable nucleic acid.

### Nanoparticle Delivery

As noted herein, those skilled in the art will be aware that a variety of administration systems have been developed to achieve effective delivery of nucleic acids into cells, including within mammalian (e.g., human) subjects.

Among such available technologies are various nanoparticle technologies including, for example, hydrogel, lipid, and/or polymer nanoparticle technologies.

In some embodiments, a nucleic acid is delivered to a subject in accordance with the present disclosure using a lipid nanoparticle. As used herein, the phrase “lipid nanoparticle” refers to a transfer vehicle comprising one or more lipids (e.g., cationic lipids, non- cationic lipids, and PEG-modified lipids). In some embodiments, lipid nanoparticles are formulated to deliver one or more copies of the nucleic acid to one or more target cells. Examples of suitable lipids include, for example, the phosphatidyl compounds (e.g., phosphatidylglycerol, phosphatidylcholine, phosphatidylserine, phosphatidylethanolamine, sphingolipids, cerebrosides, and gangliosides).

In some embodiments, a nucleic acid is delivered to a subject in accordance with the present disclosure using a polymer nanoparticle. Suitable polymers may include, for example, polyacrylates, polyalkycyanoacrylates, polylactide, polylactide- polyglycolide copolymers, polycaprolactones, dextran, albumin, gelatin, alginate, collagen, chitosan, cyclodextrins, dendrimers and polyethylenimine.

In some embodiments, lipids for use in the delivery of a nucleic acid of the present invention include those described in international patent publication WO 2010/053572, incorporated herein by reference. In certain embodiments, the compositions and methods of the invention employ a lipid nanoparticles comprising an ionizable cationic lipid described in U.S. provisional patent application 61/617,468, filed Mar. 29, 2012 (incorporated herein by reference), such as, e.g, (15Z, 18Z)-N,N-dimethyl-6-(9Z, 12Z)-octadeca-9,12-dien-1-yl)tetracosa-15,18-dien-1-amine (HGT5000), (15Z, 18Z)-N,N-dimethyl-6-((9Z, 12Z)-octadeca-9,12-dien-1- yl)tetracosa-4,15,18-trien-1-amine (HGT5001), and (15Z,18Z)-N,N-dimethyl-6-((9Z, 12Z)-octadeca-9,12-dien-1-yl)tetracosa-5,15,18-trien-1-amine (HGT5002).

In some embodiments, the lipid N-[1-(2,3-dioleyloxy)propyl]-N,N,N-trimethylammonium chloride or “DOTMA” is used. (Feigner et al. (Proc. Nat'l Acad. Sci. 84, 7413 (1987); U.S. Pat. No. 4,897,355). DOTMA can be formulated alone or can be combined with the neutral lipid, dioleoylphosphatidyl-ethanolamine or “DOPE” or other cationic or non-cationic lipids into a liposomal transfer vehicle or a lipid nanoparticle, and such liposomes can be used to enhance the delivery of nucleic acids into target cells. Other suitable lipids include, for example, 5- carboxyspermylglycinedioctadecylamide or “DOGS,” 2,3-dioleyloxy-N-[2(spermine- carboxamido)ethyl]-N,N-dimethyl-1-propanaminium or “DOSPA” (Behr et al. Proc. Nat.'l Acad. Sci. 86, 6982 (1989); U.S. Pat. Nos. 5,171,678; 5,334,761), 1,2-Dioleoyl-3-Dimethylammonium-Propane or “DODAP”, 1,2-Dioleoyl-3- Trimethylammonium-Propane or “DOTAP”. Contemplated lipids also include 1,2-distearyloxy-N,N-dimethyl-3-aminopropane or “DSDMA”, 1,2-dioleyloxy-N,N-dimethyl-3-aminopropane or “DODMA”, 1,2-dilinoleyloxy-N,N-dimethyl-3-aminopropane or “DLinDMA”, 1,2-dilinolenyloxy-N,N-dimethyl-3-aminopropane or “DLenDMA”, N-dioleyl-N,N-dimethylammonium chloride or “DODAC”, N,N-distearyl-N,N-dimethylarnmonium bromide or “DDAB”, N-(1,2-dimyristyloxyprop-3-yl)-N,N-dimethyl-N-hydroxyethyl ammonium bromide or “DMRIE”, 3-dimethylamino-2-(cholest -5-en-3-beta-oxybutan-4-oxy)-1-(cis,cis-9,12- octadecadienoxy)propane or “CLinDMA”, 2-[5′-(cholest-5-en-3-beta-oxy)-3′- oxapentoxy)-3-dimethy 1-1-(cis,cis-9′,1-2′-octadecadienoxy)propane or “CpLinDMA”, N,N-dimethyl-3,4-dioleyloxybenzylamine or “DMOBA”, 1,2-N,N′- dioleylcarbamyl-3-dimethylaminopropane or “DOcarbDAP”, 2,3-Dilinoleoyloxy-N,N-dimethylpropylamine or “DLinDAP”, 1,2-N,N′-Dilinoleylcarbamyl-3-dimethylaminopropane or “DLincarbDAP”, 1,2-Dilinoleoylcarbamyl-3-dimethylaminopropane or “DLinCDAP”, 2,2-dilinoleyl-4-dimethylaminomethyl-[1,3]-dioxolane or “DLin—DMA”, 2,2-dilinoleyl-4-dimethylaminoethyl-[1,3]-dioxolane or “DLin-K-XTC2-DMA”, and 2-(2,2-di((9Z, 12Z)-octadeca-9,12-dien-1-yl)-1,3-dioxolan-4-yl)-N,N-dimethylethanamine (DLin-KC2-DMA)) (See, WO 2010/042877; Semple et al., Nature Biotech. 28: 172-176 (2010) (Heyes, J., et al., J Controlled Release 107: 276-287 (2005); Morrissey, D V., et al., Nat. Biotechnol. 23(8): 1003-1007 (2005); PCT Publication WO2005/121348A1.), DLin-MC3-DMA (See WO2015199952A1 Tam and Cullis Pharmaceutics. 2013 Sep; 5(3): 498-507) or mixtures thereof. The use of cholesterol-based cationic lipids is also contemplated by the present invention. Such cholesterol-based cationic lipids can be used, either alone or in combination with other cationic or non-cationic lipids. Suitable cholesterol-based cationic lipids include, for example, DC-Choi (N,N-dimethyl-N-ethylcarboxamidocholesterol), 1,4-bis(3-N-oleylamino-propyl)piperazine (Gao, et al. Biochem. Biophys. Res. Comm. 179, 280 (1991); Wolf et al. BioTechniques 23, 139 (1997); U.S. Pat. No. 5,744,335), or ICE.

In some embodiments an oncolytic virus is a virus that preferentially infects and kills cancer cells. In some embodiments an oncolytic virus is a wild type virus that preferentially infects and kills cancer cells. In some embodiments an oncolytic virus is an engineered virus that preferentially infects and kills cancer cells. In some embodiments, an oncolytic virus can be a herpes virus, vaccinia virus, a vesicular stomatitis virus, a poliovirus, a reovirus, a senecavirus, an adenovirus.

### Vector Delivery

In some embodiments, a translatable nucleic acid as described herein may be delivered to a subject by administration of a nucleic acid vector that encodes and/or templates the translatable nucleic acids. In some embodiments, a useful vector may be or comprise a viral vector.

In some embodiments, a vector system (e.g., a viral vector system) may be or comprise components and/or sequences found in nature (i.e., wild type components and/or sequences); in some embodiments, a vector system may be or comprise engineered components and/or sequences (i.e., components whose sequence has been modified relative to an appropriate wild type reference and/or components that are not found together in a wild type reference but may, for example, represent an assemblage of components from a plurality of different sources).

Those skilled in the art are familiar with a variety of viral vector systems that could be useful in accordance with the present disclosure.

In some embodiments, a viral vector system may be or comprise components of a virus that preferentially infects cancer cells (e.g., an oncolytic virus). Those skilled in the art are aware of a variety of oncolytic viruses including, for example, vaccinia virus, a vesicular stomatitis virus, a poliovirus, a reovirus, a senecavirus, and adenovirus.

The present disclosure provides an insight that use of an oncolytic viral vector system may have certain advantages, for example in potentially providing a complementary mechanism of killing for tumor cells.

However, as noted herein, the degree of oncoselectivity achieved in accordance with the present disclosure renders oncoselectivity of a nucleic acid delivery vector not critical to many embodiments of the disclosure.

### Subjects

As described herein, the present disclosure provides technologies that are particularly useful in the treatment of cancer.

In some embodiments, provided technologies are applied to subjects suffering from cancer. That is, in some embodiments, a translatable nucleic acid as described herein (e.g., comprising at least one oncoselective translation sequence elements and a payload-encoding sequence) is delivered to (e.g., by administration of a composition comprising the translatable nucleic acid, or of a composition that causes the translatable nucleic acid to be generated in or by the subject).

In some embodiments, a subject has received, is receiving and/or will receive other therapy (e.g., other therapy to treat the cancer and/or one or more side effects of the cancer or its treatment). In some such embodiments, a payload is or comprises a protein that increases susceptibility of cells to the other therapy.

In some embodiments, a subject is not receiving a pharmaceutical agent that is known to cause stop codon readthrough in healthy cells. In some embodiments, a subject is not receiving aminoglycosides and/or macrolides.

In some embodiments, a subject is not receiving cystic fibrosis and/or Duchenne muscular dystrophy therapy (e.g. Ataluren or PTC124).

In some embodiments, a subject is not receiving pyronaridine tetraphosphate (anti-malarial), and potassium para-aminobenzoate (PABA, used of Peyronie's disease), experimental compounds RTC13, RTC14, and NB54, and/or herbal supplement escin.

In some embodiments, a subject is not affected by ribosomopathies such as Diamond-Blackfan anemia, Dyskeratosis congenita, Shwachman-Diamond syndrome, 5q-myelodysplastic syndrome, Treacher Collins syndrome, Cartilage-hair hypoplasia, Isolated congenital asplenia, Bowen-Conradi syndrome, North American Indian childhood cirrhosis.

### EXEMPLIFICATION

### Example 1: Exemplary Oncoselective Read-Through Motifs

The present Example describes certain exemplary read-through motifs that confer oncoselective expression (i.e., “oncoselective read-through motifs”) as described herein, as well as certain approaches to identifying and/or characterizing such motifs. As described herein, the present disclosure teaches that nucleic acid sequence elements with particular structural features (e.g., primary, secondary, and/or tertiary structural characteristics) direct selective translation in cancer cells relative to non-diseased cells, in particular by directing read-through translation specifically in cancer cells; thus, the present disclosure defines, and characterizes, describes sequence elements that are oncoselective read-through motifs.

Certain oncoselective read-through motifs described herein (i.e., that direct read-through of mRNA stop codons specifically in cancer cells relative to appropriately comparable non-cancer cells were identified and/or characterized by employing a combination of different techniques. In some embodiments, a read-through motif that confers oncoselective expression is characterized in that both (1) it shows increased ribosome occupancy in ribosome profiling (e.g., Ribo-seq) analyses, and (2) its presence correlates with elevated levels of 3′-UTR-encoded peptide detectable by LC/MS in a cancer-specific proteome and/or (3) it specifically confers increased read-through to one or more oncoselective ribosomes when included in a reporter construct.

Indeed, among other things, the present disclosure provides specific insights relating to the source of a problem with certain technologies utilized in the art for assessing translation of sequences within a transcriptome, and describes strategies that are effective to identify and/or characterize useful oncoselective read-through motifs.

For example, the present disclosure appreciates that available technologies such as Ribo-seq (ribosome profiling) are useful in providing, for example, transcriptome-wide information on ribosome locations in any given cell. The present disclosure provides an insight that, while such information (particularly in conjunction with triplet periodicity) can be used to infer translation efficiency, it is biased towards RNA sequences and structures that slow down or stall the ribosomes, and may “falsely” identify such sequences and structures as apparent read-through motifs.

The present disclosure teaches that this problem with ribosome localization analyses can be addressed, at least in part, by performing one or more complementary analyses that do not have the same biases. For example, the present disclosure appreciates that liquid chromatography-mass spectrometry (LC/MS), is free of such RNA-level artifacts; proteins or polypeptides can readily be detected with high resolution LC/MS systems such as Orbitrap. The present disclosure further appreciates the source of a problem with such LC/MS technologies, however, in that, for example, peptides with low flyability can be underrepresented or missed with an LC/MS-only approach. In addition, peptides that are not among the most abundant 20 peptides of a certain fraction of liquid chromatography are not detected.

The present disclosure demonstrates that careful selection of combinations of technologies, specifically including, for example, combination of one or more ribosome occupancy analysis technology (e.g., Ribo-seq/ribosome profiling) and one or more polypeptide analysis technologies (e.g., LC/MS, reporter polypeptides, etc), can be important to accurately and/or efficiently identify and/or characterize oncoselective read-through motif(s) as described herein.

The present Example describes use of a combination of Ribo-seq and LC/MS technologies to thoroughly interrogate read-through transcriptome in cancer and healthy cells and/or to define and/or characterize certain oncoselective read-through motifs.

To determine read-through events at the proteomic level, a mass-spectrometry-based data generation pipeline was constructed. See, e.g., Mertins et al. Nature. 2016 May 25; 534(7605): 55-62. Briefly, a list of putative peptides that can be encoded by only 3′ UTRs is prepared using a custom python script. Then, regular CDS (from SwissProt human proteome), putative small open reading frame (sORF) peptide datasets (Price2, ORF-RATER, Rp-Bp), selenoprotein datasets (selenoDB), and contaminant peptides are added to the search space. Decoy peptides were generated by MaxQuant software (version 1.6.4.0). MS-MS tandem ion spectra were analyzed and matched with the putative peptides from our search space by MaxQuant via cloud computing (AWS instance with 96 vCPU, 768 GB RAM, 4x900GB SSD). Readout files were analyzed by a custom python script to extract peptides that can only be encoded by 3′UTRs. In order to verify that the peptides cannot originate from human CDS, peptide sequences are blasted against NCBI human proteome. In addition, using publicly available LC-MS data, we interrogated whether these peptides are produced in human tumor or healthy samples, TCGA breast cancer MS proteome data and CPTAC healthy breast proteome data and draft map of human proteome data, which includes 17 healthy adult tissues, 7 fetal tissues, and 6 purified primary hematopoietic cells, respectively (Mertins et al., 2016. Nature. 534(7605):55-62; Kim et al., 2014. Nature. 509(7502):575-81). This pipeline identified a large number of peptides that can only be produced by ribosomes via stop codon read-through events.

Ribosomal position on nucleic acid sequences and genome-wide translational profiling from healthy and cancer cells was analyzed to identify transcripts having a higher number of ribosomes in the 3′UTR region which would also suggest read-through. See, e.g., Zur et al., Sci Rep. 2016 Feb 22;6:21635; Ingolia et al. Science. 2009 Apr 10;324(5924): 218-23. Briefly, mRNA regions that are protected by ribosomes are deep sequenced. The 28-32 nt reads are mapped to the transcriptome to identify the regions that are occupied by ribosomes. This analysis identified the 3′UTR region of GAPDH as having relatively low ribosomal occupancy whereas FUNDC1 and CYTH1 both had relatively high read counts for the 3′UTR.

Nucleic acid sequences corresponding to the peptide data sets identified by the mass spec pipeline were analyzed with Ribo-Seq because stop codon read-through events can be verified when translating ribosomes footprints are found in 3′UTR sequences. The Trips-Viz server tool (Michel et al, 2018 Nucleic Acids Res. 2018 Jan 4;46(D1): D823-D830; Kiniry et al, Nucleic Acids Res. 2019 Jan 8;47(D1):D847-D852) was used to check the transcripts of interest for their ribosome footprints in 24 datasets from human malignant and healthy cell lines. Also, a proprietary pipeline of bioinformatics tools and graphical user interface for Ribo-seq analyses was used. The number of reads on CDS region was compared to the reads from 3′ UTR. In order to account for the lower quantity of data from the healthy data sets, triplet periodicity cutoff for Ribo-seq analysis was set at 0.72 for cancer cells and 0.000 for healthy cells. For each transcript, if there were sufficient number of reads (>500 reads) within the CDS region to indicate an actively translated mRNA, and if there was unambiguous ribosome footprint signal within 3′UTR region that did not correspond to another downstream ORF then, that transcript was considered a read-through mRNA.

Read-through transcripts were also searched in reverse order. Read-through rates of human transcripts were determined by Ribo-seq and the mass spec dataset was checked to determine whether the top Ribo-seq hits are also present in LC/MS read-through datasets. High Ribo-seq read counts may originate from secondary structures of mRNA that impedes translation and thus may not correlate with high translation rates in cancer cells.

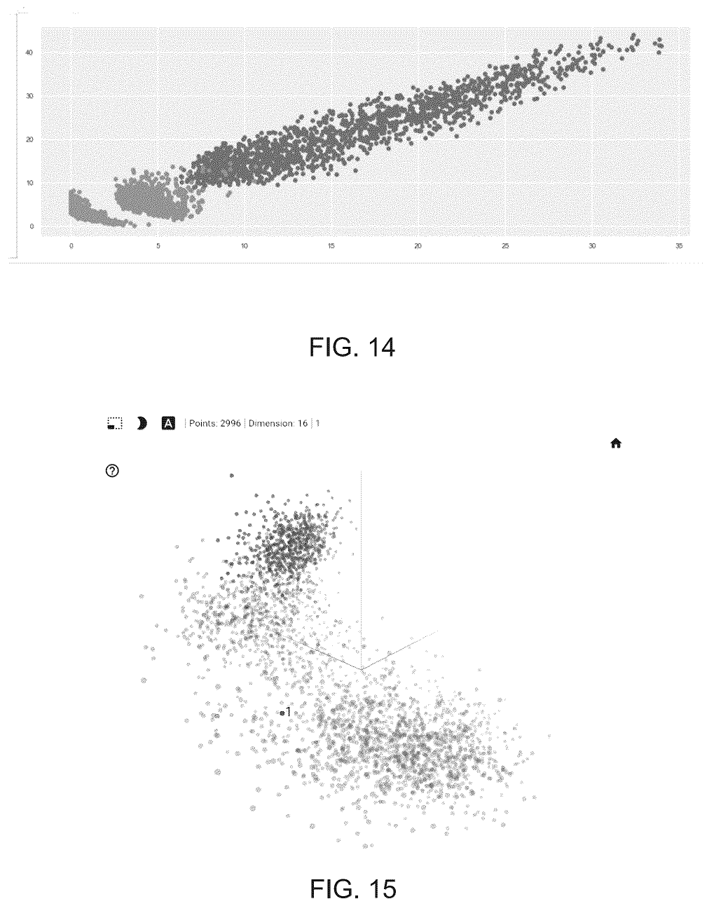

Among other things, analyses described herein identified upstream 3′UTR sequences (10 mers) of onco-selective readthrough transcripts with stop codons UAA and UAG as being more closely associated with each other compared to the 3′UTR sequences of UGA containing onco-selective readthrough trancripts. Our deep learning model, fully connected autoencoder, showed that UAA and UAG groups have very tight clusters in the latent space while UGA group has spread representation (FIGS. 14 and 15). This correlates with the efficiency of stop codons which has been reported in the literature (Loughran et al., Nucleic Acids Res. 2014; 42:8928-38).

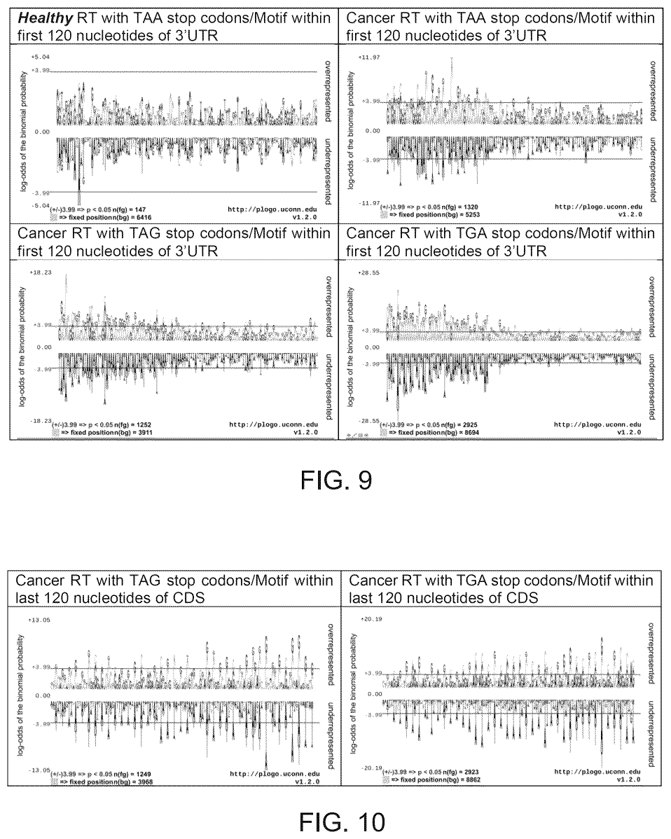

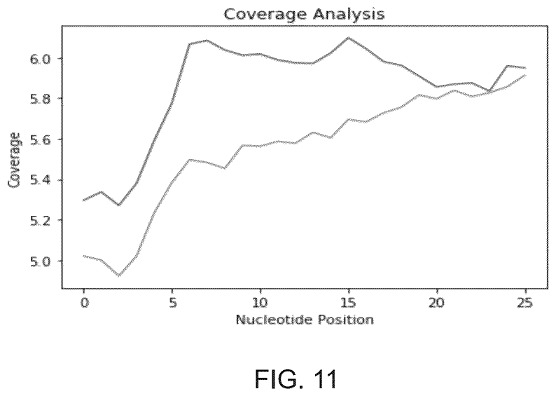

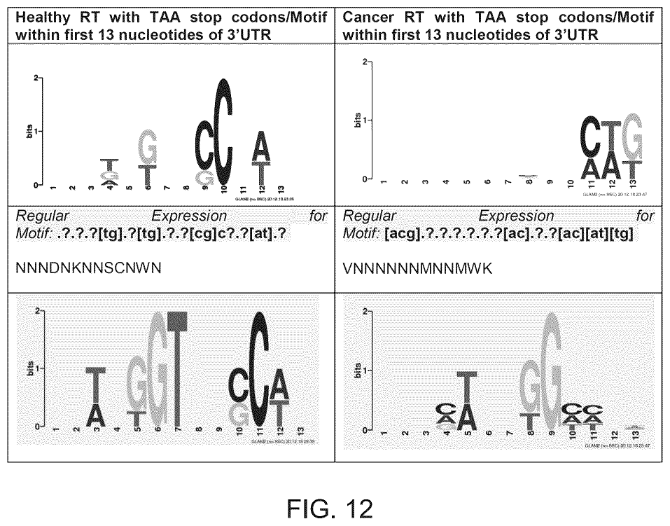

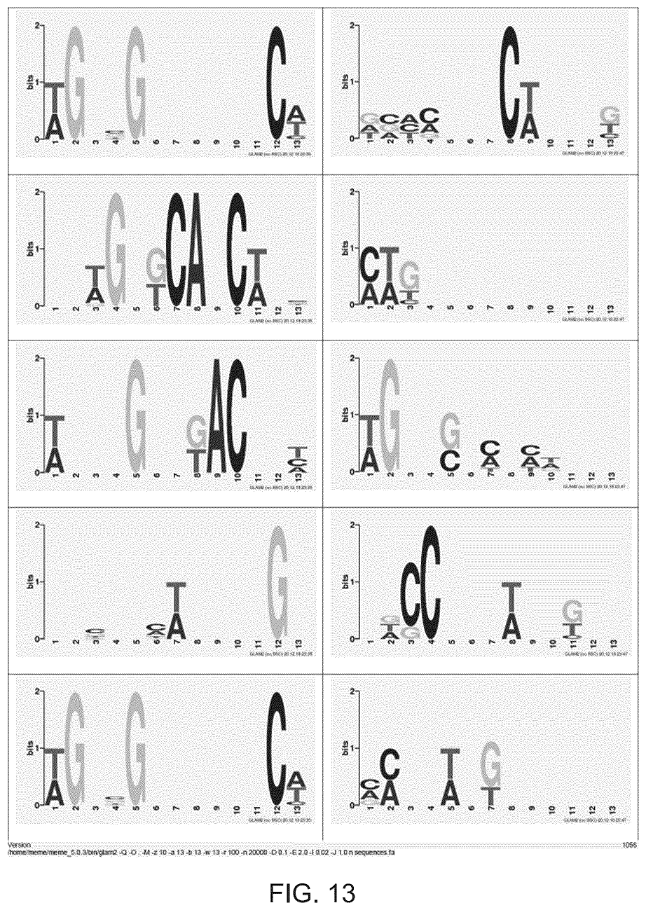

Further, nucleic acid sequences identified as having read-through stop codons were analyzed for the structural features near the canonical stop codon using algorithms including NUPACK and CoFold. Position weight matrix based sequence analysis was applied to the last 120 nucleotides of CDS and first 120 nt of 3′UTR of mRNA read-through transcripts. As shown in FIG. 9, for each of the 3 possible stop codons cancer read-through transcripts have G-C overrepresentations within the first 120 nucleotides, particularly within the first 48-50 nucleotides of the 3′UTR compared to the healthy read-through transcripts. Additionally, as shown in FIG. 10, the last 120 nucleotides of the coding sequence (CDS) of cancer read-through transcripts having read-through stop codons also have overrepresentation of G-C nucleotides compared to the healthy read-through transcripts. Healthy read-through transcipts also have G-C overrepresentations when compared to non-readthrough transcripts both in CDS and 3′UTR. CoFold analysis of the human transcriptome was performed reviewing the region around the stop codon (100 nucleotides in CDS, 100 nt in 3′UTR). This analysis showed that stop codon read-through involves higher degree of base pairing i.e. more structured regions, e.g., stem loops and bulge loops, around the stop codon, particularly within stop codons and the first 16 nucleotides of the 3′UTR sequence (FIG. 11). Cancer transcripts are even more structured around the stop codon region. This increased relative structure corresponds to, on average, 7.52 kcal/mol lower ΔG values relative to healthy read-through transcripts. Cancer transcripts have, on average. 22.5 base pairs (vs 21.6 base pairs) and 55% GC content (vs 42%) within the first 50 nucleotides of the 3′UTR.

### Example 2: Exemplary Oncoselective Read-Through Constructs

The present Example describes constructs incorporating exemplary oncoselective read-through motifs as described herein, as well as certain characterization thereof.

Constructs were made that incorporated putative onco-selective read-through motifs identified and/or characterized, for example, as described in Example 1. 10 hits from the Mass spec read-through events list that were demonstrated to have oncoselective read-through profiles in Ribo-seq analysis were selected for inclusion in an oncoselective construct. Sequences of these exemplary putative oncoselective read-through motifs are listed in Table 1

| | ENS | Sequence |
|---|---|---|
| U1 | 380715 | AAACGCAAGCAAGAGGAACAAATGGAAACTGAGCATTTTGCTCTGT |
| SEQ ID | | AAAGCATTTCAGATGCAGCTTGTGTG |
| NO. 1 | | |
| U2 | 428608 | GCGCTGACCCAGCTGATCCAGCTCTATCATCGCTTCCACCGGGTGCT |
| SEQ ID | | GTCCCAGCCGCAGCTCCGAGCCCTCCCTGCCCGGGCTGAGCTCATCA |
| NO. 2 | | ACATTCACCACCTTATGGTGGAGCTCAAGAAGCATAAGCCCAACTTC |
| | | TGATGTGCCAGAAACCGCCCTGAGATCTGCCGGTCATCTCCATGGAC |
| | | TTCTGCACCCCATTCCATACCCTTCTTCACCTGGGGTACCCCTTCCAG |
| | | TTTTCCCCTTGCTTCCCAGGCCCTTGACATGGCTTACCTGCCTTCAC |
| | | TCCCAGCACCTTGCCCAACAGGA |
| U3 | 409841 | CTTCCAGCAGAGGAGAACATGTCTAACACGTGCCTCAAAAGCACTGG |
| SEQ ID | | GGAGTTAGTAGTGCAGTGGCATCTGAAACCTGTGGAGCAGAAAGCA |
| NO. 3 | | CATGAGTCCTAATGCCCCAGCAGCTTCCGATTG |
| U4 | 274358 | ATAGTATACCCTGGAATTGCTGTATTTTTCCAGAATGCCTTCATCTTT |
| SEQ ID | | TTTGGAGGGCTGGTTTTTAAGTTTGGCCGCACTGAAGACTTATGGC |
| NO. 4 | | AGTGAACACATCTGATTTCCCACAGCACAACAGCCCTGCATGGGTTT |
| | | GTTTGTTTTTTTACTGCTCACTCCCAACCTTTTGTAATGCCATTTTC |
| | | TAAACTTATTTCTGAGTGTAGTCTCAGCTTAAAG |
| U5 | 425191 | GGTGACAAATCCATTCGTTTCCGTCCCACGCTGGTCTTCAGGGATCA |
| SEQ ID | | CCACGCTCACCTGTTCCTCAATATTTTCAGTGACATCTTAGCAGACT |
| NO. 5 | | TCAAGTAAAGAAGCCATTTCCACTACAGTGAGAAAGCCCGGATCCCA |
| | | ACAGTTGTCAAATTGATTAGTTTGCCTAATTCATGTTTTCACTTAAA |
| | | AGTATCAGAGGTG |
| U6 | 332780 | TGTCCTTTGGGCTCTGTGGAGAGCTTTAGCCTTGCACGGCGGCGCTG |
| SEQ ID | | GGAGGCATTGCCTGCCATGCCCACTGCCCGCTGCTCCTGCTCTAGTC |
| NO. 6 | | TGCAGGCTGGGCCCCGGCTGTTTGTTATTGGGGGTGTGGCCCAGGGC |
| | | CCCAGTCAAGCCGTGGAGGCACTGTGTCTGCGTGATGGGGTCTGAAG |
| | | GCTTGGTGGGAGCTGTCCACTGGAGCAGCTCATTGCCAGAGGCAGCT |
| | | ATTTCTATGGCTCCTTTTGCTGCTGAGGA |
| U7 | 360534 | ATATATACCCACATGAGCCACTTCATAAAACAATGTTTCTCTTTACCT |
| SEQ ID | | TAGCACCTCAAAATACCATGCCATTTAAAGCTTATTAAAACTCATTT |
| NO.7 | | TTGTTTTCATTATCTCAAAACTGCACTGTCAAGATGATGATGATCTT |
| | | TAAAATACACACTCAAATCAAGAAACTTAAGGTTACCTTTGTTCCCA |
| | | AATTTCATACCT |
| U8 | 450195 | GAGGTTCTCCAGGACCTTAGGTTTGATGCGGAATCTGCCGAGTGATG |
| SEQ ID | | GCGGCTCCCCAGGGATGCGCCGAGGGAGATGGGAAACGGGGCGGAT |
| NO. 8 | | GGCGCCCAGCCCAGCCCTAACTGCCAGCTGGCTGGGGTTGCGCCCCA |
| | | CTGCGCTGCTGACCTTCCTGCAGTTCCAGACACCTCCCACAATAAAG |
| | | AGCTCCTCCTCTGT |
| U9 | 337231 | GGTGAGGTCAAGGCAGGAGAGAAGAGCCTGAGCCAGCACGTGGAGG |
| SEQ ID | | CCGTGGACAAGCGGCTGGAACAGAGCCAGCCCGAGTACAAGGCGCTC |
| NO. 9 | | TTCAAAGAGATCTTCTCCAGGATCCAGAAGACCAAGGCTGACATCAA |
| | | CGCCACCAAAGTCAAGACGCACAGCAGCAAGTGACCCTTCTCCGGCC |
| | | TGCAGCCTCCC |
| U10 | 358607 | GAGGCGATCAGCGAGGTTCTCCAGGACCTTAGGTTTGATGCGGAATC |
| SEQ ID | | TGCCGAGTGATGGCGGCTCCCCAGGGATGCGCCGAGGGAGATGGGAA |
| NO. 10 | | ACGGGGCGGATGGCGCCCAGCCCAGCCCTAACTGCCAGCTGGCTGGG |
| | | GTTGCGCCCCACTGCGCTGCTGACCTTCCTGCAGTTCCAGACACCTC |
| | | CCACAATAAAGAGCTCCTCCTCTGT |

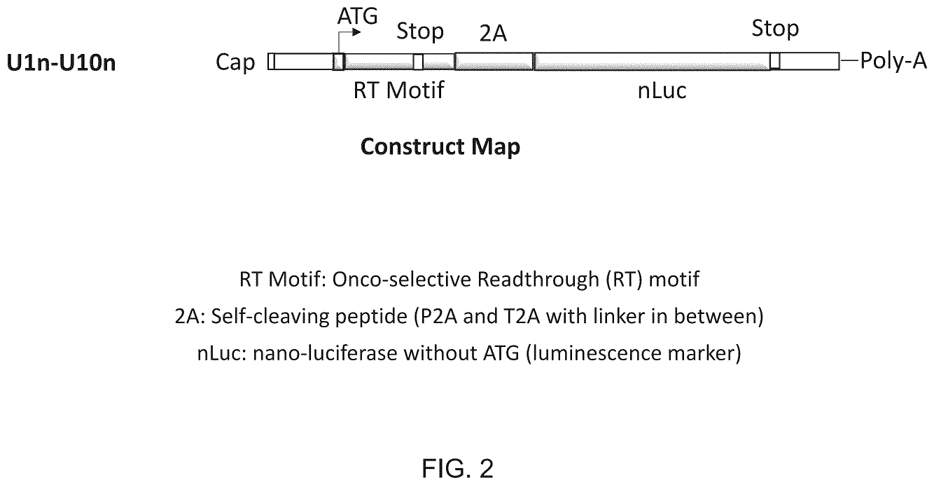

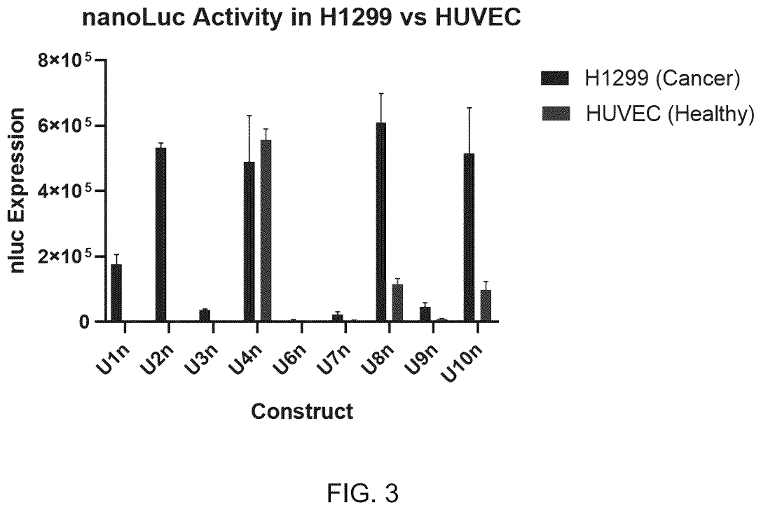

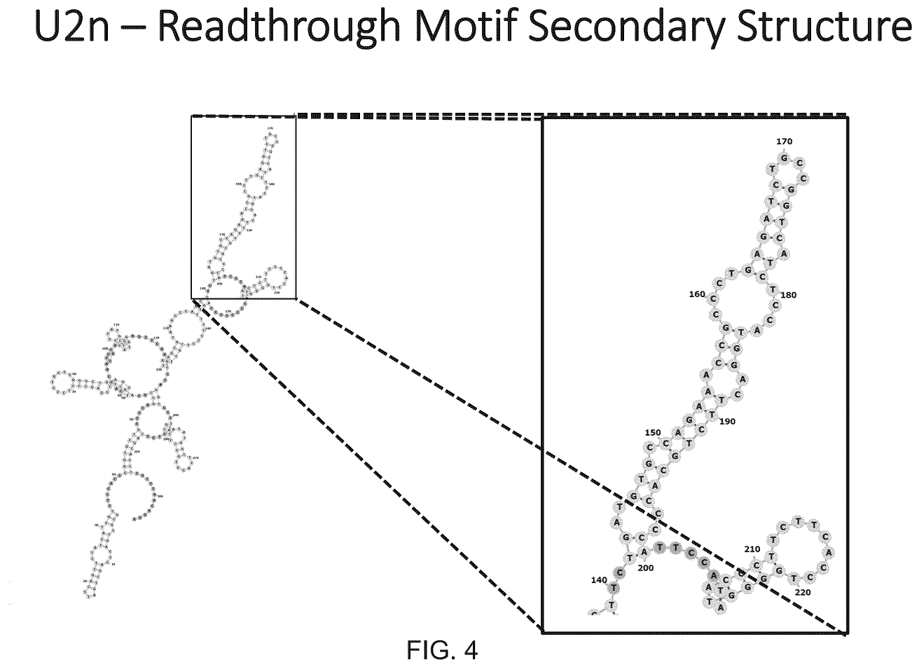

FIG. 2 shows an exemplary test construct map describing where a read-through motif was inserted in a construct encoding nano-luciferase. This particular read-through motif included a putative oncoselective read-through sequence around a stop codon; the read-through motif included a stop codon as well as the flanking sequences derived from the neighboring coding region and 3′UTR of the original gene from which the cassette is derived. FIG. 3 shows the nano-luciferase expression levels of each of the 10 constructs when transfected into either human umbilical vein endothelial cells (HUVEC) or H1299 lung cancer cells. Most read-through sequences tested showed preferred expression in cancer cells versus healthy cells. FIG. 4 shows folding of the region around the stop codon of construct U2n. There is a large stem loop structure that spans the stop codon and the 3′UTR sequence.

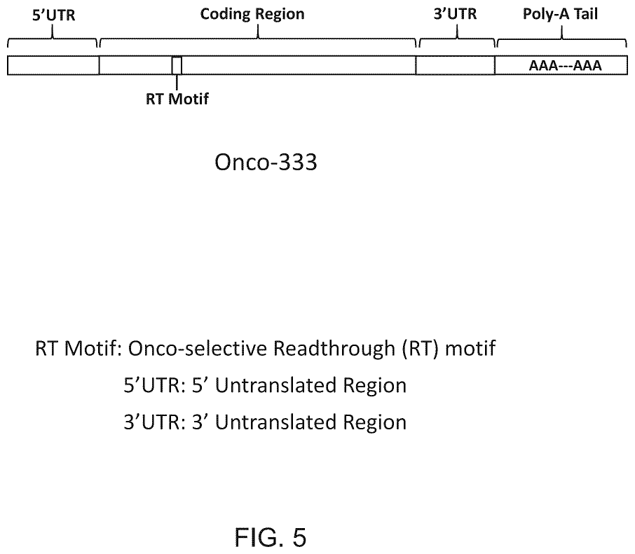

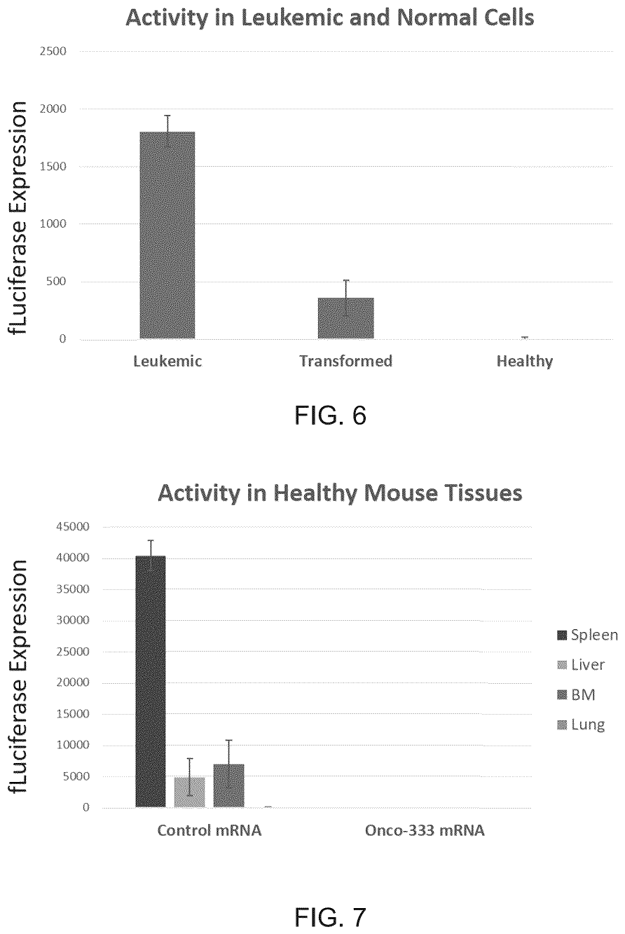

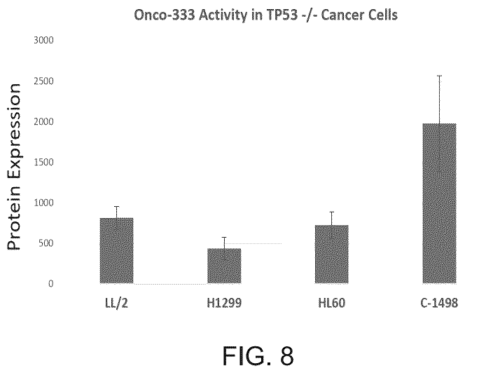

To further assess oncoselectivity of candidate and/or identified oncoselective read-through motifs as described herein, an exemplary read-through sequence was inserted into the firefly luciferase sequence to generate a construct, Onco-333, as demonstrated in FIG. 5. As can be seen with reference to FIG. 6 , this construct was expressed in leukemic K562 cells and transformed HEK293 cells but was not expressed in healthy BJ foreskin cells. Additionally, though the wild type firefly luciferase mRNA expressed in healthy mouse tissues, the firefly luciferase sequence containing the read-through sequence did not express in healthy mouse tissues (FIG. 7). As shown in FIG. 8, this construct was also tested in TP53 mutant cancer cell lines of human or murine origin, where it showed positive expression. These data demonstrate the oncoselectivity of the read-through motif.

Onco-333 fLuc sequence (UTR sequences and the poly A tail are capitalized):

(SEQ ID NO. 22)
GAAATAAGAGAGAAAAGAAGAGTAAGAAGAAATATAAGAGCCACCatgg
aagacgccaaaaacataaagaaaggcccggcgccattctatccgctgga
agatggaaccgctggatagcaactgcataaggctatgaagagatacgcc
ctggttcctggaacaattgcttttacagatgcacatatcgaggtggaca
tcacttacgctgagtacttcgaaatgtccgttcggttggcagaagctat
gaaacgatatgggctgaatacaaatcacagaatcgtcgtatgcagtgaa
aactctcttcaattctttatgccggtgttgggcgcgttatttatcggag
ttgcagttgcgcccgcgaacgacatttataatgaacgtgaattgctcaa
cagtatgggcatttcgcagcctaccgtggtgttcgtttccaaaaagggg
ttgcaaaaaattttgaacgtgcaaaaaaagctcccaatcatccaaaaaa
ttattatcatggattctaaaacggattaccagggatttcagtcgatgta
cacgttcgtcacatctcatctacctcccggttttaatgaatacgatttt
gtgccagagtccttcgatagggacaagacaattgcactgatcatgaact
cctctggatctactggtctgcctaaaggtgtcgctctgcctcatagaac
tgcctgcgtgagattctcgcatgccagagatcctatttttggcaatcaa
atcattccggatactgcgattttaagtgttgttccattccatcacggtt
ttggaatgtttactacactcggatatttgatatgtggatttcgagtcgt
cttaatgtatagatttgaagaagagctgtttctgaggagccttcaggat
tacaagattcaaagtgcgctgctggtgccaaccctattctccttcttcg
ccaaaagcactctgattgacaaatacgatttatctaatttacacgaaat
tgcttctggtggcgctcccctctctaaggaagtcggggaagcggttgcc
aagaggttccatctgccaggtatcaggcaaggatatgggctcactgaga
ctacatcagctattctgattacacccgagggggatgataaaccgggcgc
ggtcggtaaagttgttccattttttgaagcgaaggttgtggatctggat
accgggaaaacgctgggcgttaatcaaagaggcgaactgtgtgtgagag
gtcctatgattatgtccggttatgtaaacaatccggaagcgaccaacgc
cttgattgacaaggatggatggctacattctggagacatagcttactgg
gacgaagacgaacacttcttcatcgttgaccgcctgaagtctctgatta
agtacaaaggctatcaggtggctcccgctgaattggaatccatcttgct
ccaacaccccaacatcttcgacgcaggtgtcgcaggtcttcccgacgat
gacgccggtgaacttcccgccgccgttgttgttttggagcacggaaaga
cgatgacggaaaaagagatcgtggattacgtcgccagtcaagtaacaac
cgcgaaaaagttgcgcggaggagttgtgtttgtggacgaagtaccgaaa
ggtcttaccggaaaactcgacgcaagaaaaatcagagagatcctcataa
aggccaagaagggcggaaagatcgccgtgtaaGCTGCCTTCTGCGGGGC
TTGCCTTCTGGCCATGCCCTTCTTCTCTCCCTTGCACCTGTACCTCTTG
GTCTTTGAATAAAGCCTGAGTAGGAAAAAAAAAAAAAAAAAAAAAAAAA
AAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAA

Onco-333 RT motif:

(SEQ ID NO. 23)
ctatccgctggaagatggaaccgctggaTAGcaactgcataaggctatg
aagaga

dot bracket notation:

(((((((.(((.........))).))))))).....((((((((...((((((((

### Example 3: Exemplary Oncoselective Read-Through Constructs

The present Example further describes constructs incorporating exemplary oncoselective read-through motifs as described herein, as well as certain characterization thereof.

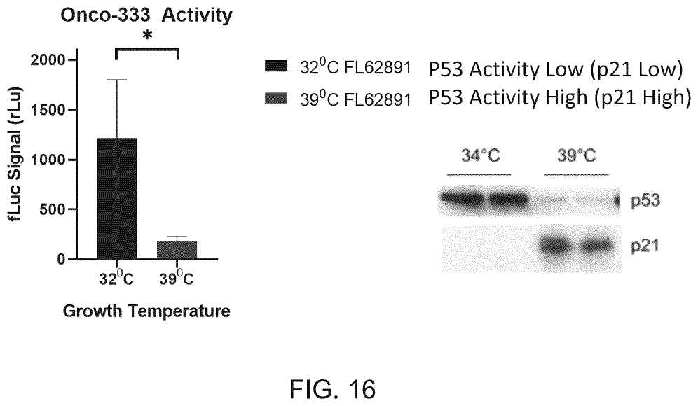

The translation activity of onco-333 was tested in FL-62891 cells under two different conditions, 32° and 39° for 7 days. Upon transfer to 39° C., a heat sensitive variant of SV40 antigen found in FL-62891 cells is inactivated, resulting in de-inhibition (i.e. activation) of p53 function and increase in p21 levels and decreasing p53 levels by a negative feedback loop on p53. Cells were seeded into white-bottom 96-well plates at 50,000 cells/well. Plates were kept at corresponding temperatures and 48 hours later cells were transfected with 200 ng onco-333 mRNA/well. Following 16 hours of incubation, firefly Luciferase activity was measured with Bright-Glo Luciferase assay on a Promega GloMax plate reader. Transfections were performed in triplicates and data is shown as mean +/−S.D. (p=0.019). Notably, the decrease in p53 activity results in increased onco-333 expression. FIG. 16.

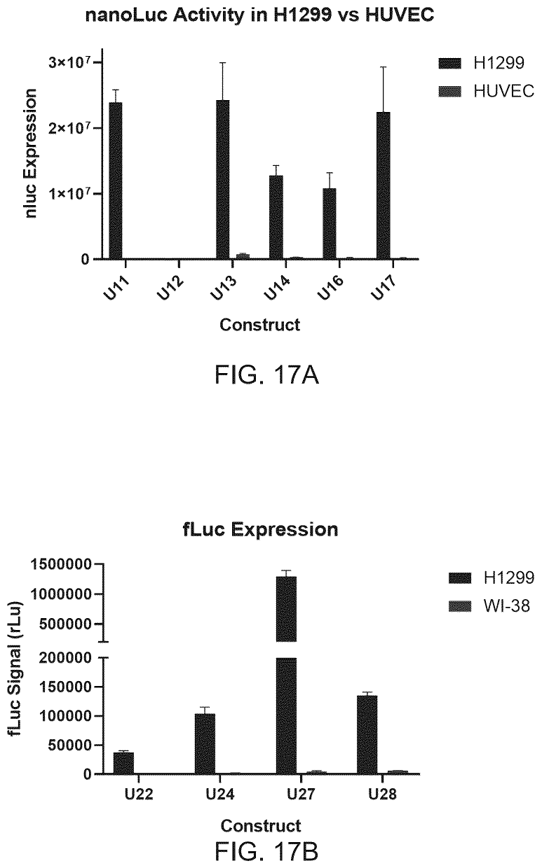

FIG. 17 shows oncoselective expression of nano-Luciferase from constructs comprising oncoselective motifs described in the present disclosure. Healthy human endothelial cells (HUVEC) and human lung cancer cells (NCI-H1299) were transfected with putative oncoselective nano-Luciferase constructs (U11-U17) and Nano-luciferase (nLuc) activity was measured 24 hours later by Promega GloMax Discover Microplate Reader. FIG. 17A shows specific expression of nano-Luciferase from the oncoselective construct in the cancer cells. Healthy human fibroblast (WI-38) and human lung cancer cells (NCI-H1299) were transfected with putative onco-selective firefly-Luciferase (fLuc) constructs and relative Luciferase activity was measured 24 hours later. FIG. 17B shows specific expression of firefly-Luciferase from the oncoselective constructs in the cancer cells. Transfections were performed in triplicates. Data shown as mean+/−standard deviation.

Sequences of these exemplary putative oncoselective read-through motifs are listed in Table 2:

| | GC | | |
|---|---|---|---|
| Construct | Content(%)* | Linear Motif* | Motif Sequence |
| U11 | 72 | VNNNNNNMNNMWK | GAGGTTCTCCAGGACCTTAGG |
| SEQ ID NO. 11 | | (SEQ ID NO: 24) | TTTGATGCGGAATCTGCCGAG |
| | | | TGATGGCGGCTCCCCAGGGAT |
| | | | GCGCCGAGGGAGATGGGAAA |
| | | | CGGGGCGGATGGCGCCCAGC |
| | | | CCAGCCCTAACTGCCAGCTGG |
| | | | CTGGGGTTGCGCCCCACTGCG |
| | | | CTGCTGACCTTCCTGCAGTTCC |
| | | | AGACACCTCCCACAATAAAGA |
| | | | GCTCCTCCTCTGT |
| U12 | 58 | WGNNSNHNHDNNN | ATCCTAGCTTCGGTGCTGGCA |
| SEQ ID NO. 12 | | (SEQ ID NO: 28) | GTGTCCCAACAGGAATACCTA |
| | | | GACAGTATGAAGAAAAACAA |
| | | | AGTGCACAGAGACCCGCCCCC |
| | | | AGACAAGAGTTGATGGAGAC |
| | | | CCAGGGATTGGACACCATCTC |
| | | | CCAACCCCAGTACTCCTGCTCT |
| | | | CCGGTGCCACCTCACCTTCTTT |
| | | | GGCTTCTTCCCTCTTGCCTCCT |
| | | | TCTGTTCTTTC |
| U13 | 50 | MWBNNNNNNNNNN | ACTGTTTCCTATGATCCTAGGA |
| SEQ ID NO. 13 | | (SEQ ID NO: 27), | AACCCACTGTGAAGAAAATTG |
| | | VMNNWNKNNNNNN | CCCCAATGATGGCCAAGACAA |
| | | (SEQ ID NO: 30) | TTAAAGCTTTCAAGAACAGAT |
| | | | TCTCCCGACGATAAACTGAGG |
| | | | ACTTGCCTTGGAAATGGAATC |
| | | | TGGGGAGGCAGGAATACAAG |
| | | | GACAGTGGGGGTTGGGGAAT |
| | | | GGAATTCTACAGGAGACTGGA |
| | | | GTCTTGCTTTGTG |
| U14 | 52 | VNNNNNNMNNMWK | TTGTCATGTGTACAGGAAATCA |
| SEQ ID NO. 14 | | (SEQ ID NO: 24), | GTGATGTGGTGCAGAGGTAGC |
| | | MWBNNNNNNNNN | CACTGTTAGCCTGGTGGGAAA |
| | | N (SEQ ID NO: 27), | ATGCACACATTTCTGAGGGGA |
| | | VMNNWNKNNNNNN | GAGGGAAAAGGACTTGTTTT |
| | | (SEQ ID NO: 30) | CCTGTGTTCTTGTTTTCAGAAA |
| | | | ATGAAAGACTCATACTTGAGT |
| | | | GTGTTTATGTG |
| U16 | 60 | VMNNWNKNNNNNN | GTGCAGCACAAAAAGCCCGC |
| SEQ ID NO. 16 | | (SEQ ID NO: 30) | CGACATCCCTCAGGGCTCCTT |
| | | | GGCCTACCTGGAGCAGGCATC |
| | | | TGCCAACATCCCTGCACCTCTG |
| | | | AAGCCAACGTGAGCAAAGGG |
| | | | CAGAGGCAGTTGGCCTATGAG |
| | | | TGGGCTGATGCGTGAGGTTG |
| | | | GCCACACATTCCTTCCTGTGG |
| | | | ACTTGACATTTTGGAAGAACT |
| | | | CTTTGCCAGATAATGAGTTCAT |
| | | | TTTAGTTTTATGCTCCCAT |
| U17 | 72 | DVHVNNNCWNNNB | TGGGGTCCCCAGTGGGGAAT |
| SEQ ID NO. 17 | | (SEQ ID NO: 26), | GAACGGGTACTTCCTCATCGA |
| | | VMNNWNKNNNNNN | GCGCGGAAAGAACATGTGTG |
| | | (SEQ ID NO: 30) | GCCTGGCTGCCTGCGCCTCCT |
| | | | ACCCCATCCCTCTGGTGTGAG |
| | | | CCGTGGCAGCCGCAGCGCAG |
| | | | ACTGGCGGAGAAGGAGAGGA |
| | | | ACGGGCAGCCTGGGCCTGGG |
| | | | TGGAAATCCTGCCCTGGAGGA |
| | | | AGTTGTGGGGAGATCCACTGG |
| | | | GACCCCCAACATTCTGCCCTCA |
| | | | CCTCTGTGCCCAGCCTGGAAA |
| | | | CCTACAGACAAGGAGG |
| U22 | 28 | MWBNNNNNNNNNN | GAAGAAACAAAGAATAAACTC |
| SEQ ID NO. 18 | | (SEQ ID NO: 27) | AAAGCCGGCAATGCCCTGGCA |
| | | | TTTTTGGGTCTTGAGAGAAAA |
| | | | CAATTTGAATGACTGAATTTAC |
| | | | TACAAAGGCAAACTTTCAAAA |
| | | | GGATATCTCTTTTTTGTTTCCA |
| | | | AATATGTATCAACAGGTATCAA |
| | | | CAAAATCCTATTTTGAACTATT |
| | | | TTACTCAGAAAAGAATATCCCA |
| | | | AATATCCCAAATTATTCATAATA |
| | | | AAAATGATTTGAAAGTGTTTT |
| | | | CATTCTTAAAA |
| U24 | 28 | MWBNNNNNNNNNN | TTTTACTGTAAGCTGTGTTCAC |
| SEQ ID NO. 19 | | (SEQ ID NO: 27) | TCTTTTATACAAATGAAGAAGT |
| | | | TGCAAAGAATACTCATTGCAG |
| | | | CAGCCTTCCTCATTATCAGAAA |
| | | | TTAAAGAAATTTCTGAATAAAT |
| | | | TGGCAGAAGAACGCAGACAG |
| | | | AAGAAGGAAACTTAAGATGTG |
| | | | CAAGGAGATTTAAGGATTTCA |
| | | | AAGAAAATAAAGGTTCTTTGT |
| | | | TTT |
| U27 | 84 | MWBNNNNNNNNNN | AAGTGAGGCTCTCCTCCCGCC |
| SEQ ID NO. 20 | | (SEQ ID NO: 27), | CCGCCCCTCCCACGCCTCACC |
| | | VNNNNNNMNNMWK | AGCCCCCCGCGCGCCCACCCT |
| | | (SEQ ID NO: 24) | CCGGCGGGTGACAGCTCCGG |
| | | | GATCAGCAACCCTTCCTGCTG |
| | | | CTGCTACTGCTGCTGCTGCTGC |
| | | | CGCCGCCGCCGCCGCCGCTGC |
| | | | CCTTGGGTCCCCCCGAGTCTC |
| | | | CGGGACTGCCCTCTCGACTGT |
| | | | CAGTGGGGCAGCCTCTCCGAC |
| | | | TCTGCACCCGCCTCGACCTCCC |
| | | | CACCCGCTCCCACACCCCTGT |
| | | | GCCCTCATGTGGAGCCTAAGA |
| | | | GAACAGAACAGGCCGTGAAG |
| | | | CCAGCAGAGAAA |
| U28 | | | CCCCGCCCTCCCAGTTTCCGC |
| SEQ ID NO. 21 | | | GCGCCTCTTTGGCAGCTGGTC |
| | | | ACATGGTGAGGGTGGGGGTG |
| | | | AGGGGGCCTCTCTAGCTTGCG |
| | | | GCCTGTGTCTATGGTCGGGCC |
| | | | CTCTGCGTCCAGCTGCTCCGG |
| | | | ACCGAGCTCGGGTGTATGGG |
| | | | GCCGTAGGAACCGGCTCCGG |
| | | | GGCCCCGATAACGGGCCGCCC |
| | | | CCACAGCACCCCGGGCTGGC |
| | | | GTGAGGGTCTCCCTTGATCTG |
| | | | AGA |

*determined for the first 50 nucleotides of the oncoselective construct.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. An engineered nucleic acid whose nucleotide sequence includes a sequence element that is or is a complement of an oncoselective translation sequence element.

2. The engineered nucleic acid of claim 1, wherein the engineered nucleic acid's nucleotide sequence includes an open reading frame or complement thereof.

3. The engineered nucleic acid of claim 2, wherein the oncoselective translation sequence element is or comprises an oncoselective read-through motif within or upstream of the open reading frame.

4. The engineered nucleic acid of claim 3 wherein the oncoselective readthrough motif comprises an upstream flanking sequence, a stop codon, and a downstream flanking sequence.

5. The engineered nucleic acid of claim 3, wherein the oncoselective readthrough motif comprises a sequence selected from the group comprising: VNNNNNNMNNMWK, NNNVWNNKGHHNH, DVHVNNNCWNNNB, MWBNNNNNNNNNN, WGNNSNHNHDNNN, VNNNNNNMNNMWK or VMNNWNKNNNNNN, wherein V stands for A, C or G, M stands for A or C, W stands for A or T/U, K stands for G or T/U, H stands for A, C or T/U, D stands for A,G or T/U, B stands for C, G or T/U, S stands for G or C, N stands for any nucleotide, within the region that spans the readthrough stop codon and the first 14 nucleotides of the downstream flanking sequence.

6. The engineered nucleic acid of claim 3, wherein the oncoselective read through motif comprises a stem loop; a bulge loop, a pseudoknot, or a combination thereof within the first 50 nucleotides of the downstream flanking sequence and part of this stem loop located preferably within stop codon and the first 16 nucleotides of the downstream flanking sequence, or a combination thereof.

7. The engineered nucleic acid of claim 6, wherein the stem loop comprises more than 20 base paired nucleotides within first 50 nucleotides of the downstream flanking sequence.

8. (canceled)

9. (canceled)

10. The engineered nucleic acid of claim 2, wherein the open reading frame encodes a suicide protein.

11. The engineered nucleic acid of claim 10, wherein the suicide protein induces necroptosis.

12. The engineered nucleic acid of claim 11, wherein the suicide protein is constitutively active MLKL.

13. The engineered nucleic acid of claim 10, wherein the suicide protein induces pyroptosis.

14. The engineered nucleic acid of claim 13, wherein the suicide protein is constitutively active gasdermin D.

15. (canceled)

16. A nucleic acid whose sequence includes an open reading frame, or a complement thereof, into or before which an oncoselective read-through motif has been engineered, wherein the open reading frame encodes a payload protein selected from the group consisting of a suicide protein, cell surface antigen, an antibody agent, a toxin, a genetic modification protein, or a viral replication protein.

17. A pharmaceutical composition comprising the nucleic acid of claim 1.

18. The pharmaceutical composition of claim 17, wherein the pharmaceutical composition comprises nanoparticles.

19-22. (canceled)

23. A method of treating cancer in a subject, wherein the method comprises administering a therapeutically effective amount of the engineered nucleic acid of claim 1 or the pharmaceutical composition of claim 17.

24. The method of claim 23, wherein the cancer in the subject comprises oncogenic ribosomes.

25-30. (canceled)

31. An oncoselective translation sequence element comprising a read-through consensus sequence, sequence with high G-C content; a codon encoding proline; a stem loop; a bulge loop, a pseudoknot or a combination thereof.

32. A method of identifying an onco-selective nucleic acid sequences the method comprising transcriptome-wide translatome analysis.

33. The method of claim 32, wherein the method comprises Ribo-seq and LC/MS-based proteomics pipelines.


SEQUENCE LISTING (32 sequences)

SEQ ID NO: 1 - DNA, 72 bp, Artificial Sequence
  Description of Artificial Sequence: Synthetic oligonucleotide
aaacgcaagcaagaggaacaaatggaaactgagcattttgctctgtaaagcatttcagatgcagcttgtgtg

SEQ ID NO: 2 - DNA, 306 bp, Artificial Sequence
  Description of Artificial Sequence: Synthetic polynucleotide
gcgctgacccagctgatccagctctatcatcgcttccaccgggtgctgtcccagccgcagctccgagccctccctgcccgggctgagctcatcaacattcaccaccttatggtggagctcaagaagcataagcccaacttctgatgtgccagaaaccgccctgagatctgccggtcatctccatggacttctgcaccccattccatacccttcttcacctggggtaccccttccagttttccccttgcttcccaggcccttgacatggcttacctgccttcactcccagcaccttgcccaacagga

SEQ ID NO: 3 - DNA, 126 bp, Artificial Sequence
  Description of Artificial Sequence: Synthetic polynucleotide
cttccagcagaggagaacatgtctaacacgtgcctcaaaagcactggggagttagtagtgcagtggcatctgaaacctgtggagcagaaagcacatgagtcctaatgccccagcagcttccgattg

SEQ ID NO: 4 - DNA, 222 bp, Artificial Sequence
  Description of Artificial Sequence: Synthetic polynucleotide
atagtataccctggaattgctgtatttttccagaatgccttcatcttttttggagggctggtttttaagtttggccgcactgaagacttatggcagtgaacacatctgatttcccacagcacaacagccctgcatgggtttgtttgtttttttactgctcactcccaaccttttgtaatgccattttctaaacttatttctgagtgtagtctcagcttaaag

SEQ ID NO: 5 - DNA, 201 bp, Artificial Sequence
  Description of Artificial Sequence: Synthetic polynucleotide
ggtgacaaatccattcgtttccgtcccacgctggtcttcagggatcaccacgctcacctgttcctcaatattttcagtgacatcttagcagacttcaagtaaagaagccatttccactacagtgagaaagcccggatcccaacagttgtcaaattgattagtttgcctaattcatgttttcacttaaaagtatcagaggtg

SEQ ID NO: 6 - DNA, 264 bp, Artificial Sequence
  Description of Artificial Sequence: Synthetic polynucleotide
tgtcctttgggctctgtggagagctttagccttgcacggcggcgctgggaggcattgcctgccatgcccactgcccgctgctcctgctctagtctgcaggctgggccccggctgtttgttattgggggtgtggcccagggccccagtcaagccgtggaggcactgtgtctgcgtgatggggtctgaaggcttggtgggagctgtccactggagcagctcattgccagaggcagctatttctatggctccttttgctgctgagga

SEQ ID NO: 7 - DNA, 201 bp, Artificial Sequence
  Description of Artificial Sequence: Synthetic polynucleotide
atatatacccacatgagccacttcataaaacaatgtttctctttaccttagcacctcaaaataccatgccatttaaagcttattaaaactcatttttgttttcattatctcaaaactgcactgtcaagatgatgatgatctttaaaatacacactcaaatcaagaaacttaaggttacctttgttcccaaatttcatacct

SEQ ID NO: 8 - DNA, 201 bp, Artificial Sequence
  Description of Artificial Sequence: Synthetic polynucleotide
gaggttctccaggaccttaggtttgatgcggaatctgccgagtgatggcggctccccagggatgcgccgagggagatgggaaacggggcggatggcgcccagcccagccctaactgccagctggctggggttgcgccccactgcgctgctgaccttcctgcagttccagacacctcccacaataaagagctcctcctctgt

SEQ ID NO: 9 - DNA, 198 bp, Artificial Sequence
  Description of Artificial Sequence: Synthetic polynucleotide
ggtgaggtcaaggcaggagagaagagcctgagccagcacgtggaggccgtggacaagcggctggaacagagccagcccgagtacaaggcgctcttcaaagagatcttctccaggatccagaagaccaaggctgacatcaacgccaccaaagtcaagacgcacagcagcaagtgacccttctccggcctgcagcctccc

SEQ ID NO: 10 - DNA, 213 bp, Artificial Sequence
  Description of Artificial Sequence: Synthetic polynucleotide
gaggcgatcagcgaggttctccaggaccttaggtttgatgcggaatctgccgagtgatggcggctccccagggatgcgccgagggagatgggaaacggggcggatggcgcccagcccagccctaactgccagctggctggggttgcgccccactgcgctgctgaccttcctgcagttccagacacctcccacaataaagagctcctcctctgt

SEQ ID NO: 11 - DNA, 201 bp, Artificial Sequence; identical to SEQ ID NO: 8
  Description of Artificial Sequence: Synthetic polynucleotide

SEQ ID NO: 12 - DNA, 201 bp, Artificial Sequence
  Description of Artificial Sequence: Synthetic polynucleotide
atcctagcttcggtgctggcagtgtcccaacaggaatacctagacagtatgaagaaaaacaaagtgcacagagacccgcccccagacaagagttgatggagacccagggattggacaccatctcccaaccccagtactcctgctctccggtgccacctcaccttctttggcttcttccctcttgcctccttctgttctttc

SEQ ID NO: 13 - DNA, 201 bp, Artificial Sequence
  Description of Artificial Sequence: Synthetic polynucleotide
actgtttcctatgatcctaggaaacccactgtgaagaaaattgccccaatgatggccaagacaattaaagctttcaagaacagattctcccgacgataaactgaggacttgccttggaaatggaatctggggaggcaggaatacaaggacagtgggggttggggaatggaattctacaggagactggagtcttgctttgtg

SEQ ID NO: 14 - DNA, 159 bp, Artificial Sequence
  Description of Artificial Sequence: Synthetic polynucleotide
ttgtcatgtgtacaggaaatcagtgatgtggtgcagaggtagccactgttagcctggtgggaaaatgcacacatttctgaggggagagggaaaaggacttgttttcctgtgttcttgttttcagaaaatgaaagactcatacttgagtgtgtttatgtg

SEQ ID NO: 15 - skipped

SEQ ID NO: 16 - DNA, 228 bp, Artificial Sequence
  Description of Artificial Sequence: Synthetic polynucleotide
gtgcagcacaaaaagcccgccgacatccctcagggctccttggcctacctggagcaggcatctgccaacatccctgcacctctgaagccaacgtgagcaaagggcagaggcagttggcctatgagtgggctgatgcgtgaggttggccacacattccttcctgtggacttgacattttggaagaactctttgccagataatgagttcattttagttttatgctcccat

SEQ ID NO: 17 - DNA, 264 bp, Artificial Sequence
  Description of Artificial Sequence: Synthetic polynucleotide
tggggtccccagtggggaatgaacgggtacttcctcatcgagcgcggaaagaacatgtgtggcctggctgcctgcgcctcctaccccatccctctggtgtgagccgtggcagccgcagcgcagactggcggagaaggagaggaacgggcagcctgggcctgggtggaaatcctgccctggaggaagttgtggggagatccactgggacccccaacattctgccctcacctctgtgcccagcctggaaacctacagacaaggagg

SEQ ID NO: 18 - DNA, 249 bp, Artificial Sequence
  Description of Artificial Sequence: Synthetic polynucleotide
gaagaaacaaagaataaactcaaagccggcaatgccctggcatttttgggtcttgagagaaaacaatttgaatgactgaatttactacaaaggcaaactttcaaaaggatatctcttttttgtttccaaatatgtatcaacaggtatcaacaaaatcctattttgaactattttactcagaaaagaatatcccaaatatcccaaattattcataataaaaatgatttgaaagtgttttcattcttaaaa

SEQ ID NO: 19 - DNA, 195 bp, Artificial Sequence
  Description of Artificial Sequence: Synthetic polynucleotide
ttttactgtaagctgtgttcactcttttatacaaatgaagaagttgcaaagaatactcattgcagcagccttcctcattatcagaaattaaagaaatttctgaataaattggcagaagaacgcagacagaagaaggaaacttaagatgtgcaaggagatttaaggatttcaaagaaaataaaggttctttgtttt

SEQ ID NO: 20 - DNA, 306 bp, Artificial Sequence
  Description of Artificial Sequence: Synthetic polynucleotide
aagtgaggctctcctcccgccccgcccctcccacgcctcaccagccccccgcgcgcccaccctccggcgggtgacagctccgggatcagcaacccttcctgctgctgctactgctgctgctgctgccgccgccgccgccgccgctgcccttgggtccccccgagtctccgggactgccctctcgactgtcagtggggcagcctctccgactctgcacccgcctcgacctccccacccgctcccacacccctgtgccctcatgtggagcctaagagaacagaacaggccgtgaagccagcagagaaa

SEQ ID NO: 21 - DNA, 230 bp, Artificial Sequence
  Description of Artificial Sequence: Synthetic polynucleotide
ccccgccctcccagtttccgcgcgcctctttggcagctggtcacatggtgagggtgggggtgagggggcctctctagcttgcggcctgtgtctatggtcgggccctctgcgtccagctgctccggaccgagctcgggtgtatggggccgtaggaaccggctccggggccccgataacgggccgcccccacagcaccccgggctggcgtgagggtctcccttgatctgaga

SEQ ID NO: 22 - DNA, 1908 bp, Artificial Sequence
  Description of Artificial Sequence: Synthetic polynucleotide
gaaataagagagaaaagaagagtaagaagaaatataagagccaccatggaagacgccaaaaacataaagaaaggcccggcgccattctatccgctggaagatggaaccgctggatagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaagctgccttctgcggggcttgccttctggccatgcccttcttctctcccttgcacctgtacctcttggtctttgaataaagcctgagtaggaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 23 - DNA, 55 bp, Artificial Sequence
  Description of Artificial Sequence: Synthetic oligonucleotide
ctatccgctggaagatggaaccgctggatagcaactgcataaggctatgaagaga

SEQ ID NO: 24 - DNA, 13 bp, Artificial Sequence
  Description of Artificial Sequence: Synthetic oligonucleotide
  modified_base (2)..(7): a, c, t, g, unknown or other
  modified_base (9)..(10): a, c, t, g, unknown or other
vnnnnnnmnnmwk

SEQ ID NO: 25 - DNA, 13 bp, Artificial Sequence
  Description of Artificial Sequence: Synthetic oligonucleotide
  modified_base (1)..(3): a, c, t, g, unknown or other
  modified_base (6)..(7): a, c, t, g, unknown or other
  modified_base (12)..(12): a, c, t, g, unknown or other
nnnvwnnkghhnh

SEQ ID NO: 26 - DNA, 13 bp, Artificial Sequence
  Description of Artificial Sequence: Synthetic oligonucleotide
  modified_base (5)..(7): a, c, t, g, unknown or other
  modified_base (10)..(12): a, c, t, g, unknown or other
dvhvnnncwnnnb

SEQ ID NO: 27 - DNA, 13 bp, Artificial Sequence
  Description of Artificial Sequence: Synthetic oligonucleotide
  modified_base (4)..(13): a, c, t, g, unknown or other
mwbnnnnnnnnnn

SEQ ID NO: 28 - DNA, 13 bp, Artificial Sequence
  Description of Artificial Sequence: Synthetic oligonucleotide
  modified_base (3)..(4): a, c, t, g, unknown or other
  modified_base (6)..(6): a, c, t, g, unknown or other
  modified_base (8)..(8): a, c, t, g, unknown or other
  modified_base (11)..(13): a, c, t, g, unknown or other
wgnnsnhnhdnnn

SEQ ID NO: 29 - DNA, 13 bp, Artificial Sequence
  Description of Artificial Sequence: Synthetic oligonucleotide
  modified_base (2)..(7): a, c, t, g, unknown or other
  modified_base (9)..(10): a, c, t, g, unknown or other
vnnnnnnmnnmwk

SEQ ID NO: 30 - DNA, 13 bp, Artificial Sequence
  Description of Artificial Sequence: Synthetic oligonucleotide
  modified_base (3)..(4): a, c, t, g, unknown or other
  modified_base (6)..(6): a, c, t, g, unknown or other
  modified_base (8)..(13): a, c, t, g, unknown or other
vmnnwnknnnnnn

SEQ ID NO: 31 - DNA, 88 bp, Artificial Sequence
  Description of Artificial Sequence: Synthetic oligonucleotide
ttctgatgtgccagaaaccgccctgagatctgccggtcatctccatggacttctgcaccccattccatacccttcttcacctggggta

SEQ ID NO: 32 - DNA, 13 bp, Artificial Sequence
  Description of Artificial Sequence: Synthetic oligonucleotide
  modified_base (1)..(3): a, c, t, g, unknown or other
  modified_base (5)..(5): a, c, t, g, unknown or other
  modified_base (7)..(8): a, c, t, g, unknown or other
  modified_base (11)..(11): a, c, t, g, unknown or other
  modified_base (13)..(13): a, c, t, g, unknown or other
nnndnknnscnwn